(19) United States
(12) Patent Application Publication
(10) Pub. No.: US 2026/0176238 A1
(43) Pub. Date: Jun. 25, 2026

(54) METHOD FOR PRODUCING ALKYLSILYLOXY-SUBSTITUTED BENZYLAMINE COMPOUND

(71) Applicant: SEKISUI MEDICAL CO., LTD., Tokyo (JP)

(72) Inventor: Shou ISHIKAWA, Chuo-ku (JP)

(73) Assignee: SEKISUI MEDICAL CO., LTD., Tokyo (JP)

(21) Appl. No.: 19/105,613

(22) PCT Filed: Sep. 27, 2023
(86) PCT No.: PCT/JP2023/035097

§ 371 Date: Feb. 21, 2025

(30) Foreign Application Priority Data
Sep. 28, 2022 (JP) 2022-155408

(51) Int. Cl. C07D 233/64; C07D 213/56; C07D 471/04; C07D 487/04

(52) U.S. Cl. CPC C07D 233/64; C07D 213/56; C07D 471/04; C07D 487/04

(57) ABSTRACT

A method for producing a ketimine compound of formula (3)

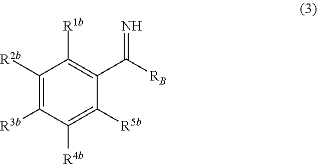

in which one to five of R^{1b} to R^{5b} each represent hydroxyalkyloxy having 1-16 carbon atoms, the remaining groups each represent hydrogen or halogen, for example, and R_{B} represents hydrogen, hydroxy, or alkoxy having 1-6 carbon atoms, for example),
wherein a silazane compound is subjected to a reaction with a benzoyl compound of formula (2)

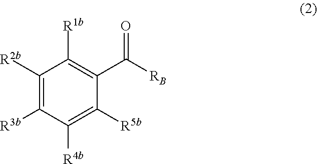

in which R^{1b} to R^{5b} and R_{B} are as described above)), and then the resulting product is subjected to a reaction with an acid or a base.

### TECHNICAL FIELD

The present invention relates to a method for producing an alkylsilyloxy-substituted benzylamine compound.

### BACKGROUND ART

In liquid phase peptide synthesis, a liquid phase peptide synthesis carrier (Tag) has been reported. Since the liquid phase peptide synthesis carrier (Tag) is a highly hydrophobic compound, solubility thereof in an organic solvent can be largely improved by bonding a highly hydrophilic amino acid, peptide, or amino acid amide (hereinafter, also referred to as an amino acid etc.) to the carrier. Therefore, a peptide elongation reaction with the carrier being bonded to an amino acid etc. has an advantage that the amino acid etc. bonded to the carrier can be easily purified by liquid-liquid separation by dissolving the amino acid etc. bonded to the carrier in an organic layer and by dissolving unnecessary components such as a surplus raw material amino acid used in the peptide elongation reaction, decomposition products thereof, and compounds produced as by-products when a protecting group of the raw material amino acids is deprotected, for example, in an aqueous layer.

Among the liquid phase peptide synthesis carriers (Tag), the liquid phase peptide synthesis carriers (Tag) described in Patent Literatures 1 to 7 and Non Patent Literature 1 each have a structure in which one or more alkyloxy side chains which each have 1-16 carbon atoms and to which an alkylsilyloxy group is bonded are bonded to a core such as a benzyl skeleton, a diphenylmethane skeleton, or a xanthene skeleton. These liquid phase peptide synthesis carriers (Tag) exquisitely control hydrophobicity of the entire liquid phase peptide synthesis carrier (Tag) by the side chain structure to which an alkylsilyloxy group is bonded, and are particularly useful as liquid phase peptide synthesis carriers (Tag).

### CITATION LIST

### Patent Literature

Patent Literature 1: JP 6116782 B2
Patent Literature 2: JP 6201076 B2
Patent Literature 3: JP 6283774 B2
Patent Literature 4: JP 6283775 B2
Patent Literature 5: JP 6322350 B2
Patent Literature 6: JP 6393857 B2
Patent Literature 7: JP 6531235 B2

### Non Patent Literature

Non Patent Literature 1: Molecules 2021, 26, 3497-3505.

### SUMMARY OF INVENTION

### Technical Problem

A method for producing these liquid phase peptide synthesis carriers (Tag) is disclosed in Patent Literatures 1 to 7. However, in this conventional method, Br—(CH₂)₁₁—O-TIPS, which is one of raw materials in the production process, is oily, has poor stability, and is difficult to handle. In addition, the raw material and an intermediate compound have similar property of being oily, which makes it difficult to separate these liquids. Therefore, the raw material or a decomposition product of the raw material is likely to be contaminated into the target product, and it is difficult to improve purity of the liquid phase peptide synthesis carrier (Tag) itself only by an ordinary industrial operation. Therefore, there is an issue that a complicated operation such as precision purification by column chromatography is required.

As described in Citation List, there is also an issue in converting an alkylsilyloxy-substituted benzyl compound into an alkylsilyloxy-substituted benzylamine compound. It has been revealed by the present inventors that stability of the alkylsilyloxy-substituted benzyl compound as an intermediate to an acid is slightly poor, and mainly two types of side reactions proceed during storage of the alkylsilyloxy-substituted benzyl compound or in a step of conversion into an amine. One is a side reaction in which a hydroxy group moiety of the benzyl compound is decomposed and the alkylsilyloxy-substituted benzyl compound is dimerized. This dimer has similar physical properties to the alkylsilyloxy-substituted benzylamine compound as a target product, and it is difficult to separate the dimer from the alkylsilyloxy-substituted benzylamine compound. The other one is a side reaction in which an alkylsilyl group is deprotected in an alkylsilyloxy group. It is also difficult to separate the dealkylsilyl body from the alkylsilyloxy-substituted benzylamine compound.

Therefore, an objective of the present invention is to provide an industrial method for producing an alkylsilyloxy-substituted benzylamine compound, in which impurities are easily removed and the method does not pass through an alkylsilyloxy-substituted benzyl compound that is an acid-labile intermediate compound.

### Solution to Problem

Therefore, the present inventors found an industrial method for producing an alkylsilyloxy-substituted benzylamine compound by introducing a hydroxyalkyloxy group into a compound having a benzoyl skeleton, a diphenyl ketone skeleton (benzophenone skeleton), or a xanthone skeleton, then causing a disilazane compound to react with the hydroxyalkyloxy-substituted body, then causing an acid or a base to react with the resulting product to deprotect a trisubstituted silyl group on a hydroxyl group, thereby obtaining an intermediate compound in which a carbonyl group bonded to each skeleton of the hydroxyalkyloxy-substituted body is converted into an imino group, then converting the imino group into a 9-fluorenylmethyloxycarbonyl-protected amino group, and then introducing an alkylsilyl group into a hydroxyl group. The method of the present invention does not require to use Br—(CH₂)₁₁—O-TIPS, which is a raw material having an issue of poor stability and difficulty in separation from an intermediate compound in the conventional method. In addition, since the intermediate compound is a solid, the raw material, a decomposition product of the raw material, and impurities generated or carried over in other systems can be easily removed by filtration or crystallization operation, for example. Further, a target product can be obtained with high purity and high yield without passing through an alkylsilyloxy-substituted benzyl compound, which is an acid-labile intermediate. As described above, the present inventors found that an alkylsilyloxy-substituted benzylamine compound can be industrially advantageously obtained, and have completed the present invention.

In the present invention, the term “solid” refers to both a solid having a crystal structure and an amorphous-like solid.

That is, the present invention provides the following [1] to [7].

[1]A method for producing a ketimine compound of general formula (3):

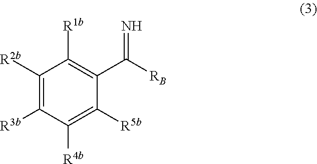

(in which one to five of R^{1b} to R^{5b} each represent a hydroxyalkyloxy group having 1-16 carbon atoms, the remaining groups each represent a hydrogen atom, a halogen atom, an alkyl group having 1-4 carbon atoms, or an alkoxy group having 1-4 carbon atoms, and
R_{B} represents a hydrogen atom, a hydroxyl group, an alkoxy group having 1-6 carbon atoms, or a group of the following formula:

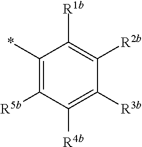

(one to five of R^{1b} to R^{5b} each represent a hydroxyalkyloxy group having 1-16 carbon atoms, and the remaining groups each represent a hydrogen atom, a halogen atom, an alkyl group having 1-4 carbon atoms, or an alkoxy group having 1-4 carbon atoms, or when R_{B} represents a structure of this formula, R^{5b} may form an ether bond (—O—) together with R^{5b} in formula (3). * represents a bonding site to R_{B}.)),
wherein a silazane compound is subjected to a reaction with a benzoyl compound of general formula (2):

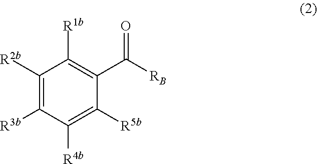

(in which one to five of R^{1b} to R^{5b} each represent a hydroxyalkyloxy group having 1-16 carbon atoms, the remaining groups each represent a hydrogen atom, a halogen atom, an alkyl group having 1-4 carbon atoms, or an alkoxy group having 1-4 carbon atoms, and
R_{B} represents a hydrogen atom, a hydroxy group, an alkoxy group having 1-6 carbon atoms, or a group of

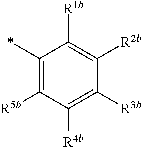

(one to five of R^{1b} to R^{5b} each represent a hydroxyalkyloxy group having 1-16 carbon atoms, and the remaining groups each represent a hydrogen atom, a halogen atom, an alkyl group having 1-4 carbon atoms, or an alkoxy group having 1-4 carbon atoms, or when Rp represents a structure of this formula, R^{5b} may form an ether bond (—O—) together with R^{5b} in formula (2). * represents a bonding site to R_{B}.)),
and then the resulting product is subjected to a reaction with an acid or a base.

[2]A method for producing a benzylamine compound of general formula (4):

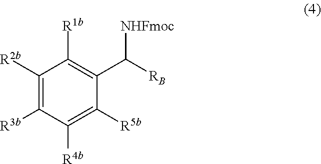

(in which one to five of R^{1b} to R^{5b} each represent a hydroxyalkyloxy group having 1-16 carbon atoms, the remaining groups each represent a hydrogen atom, a halogen atom, an alkyl group having 1-4 carbon atoms, or an alkoxy group having 1-4 carbon atoms, and
R_{B} represents a hydrogen atom, a hydroxy group, an alkoxy group having 1-6 carbon atoms, or a group of the following formula:

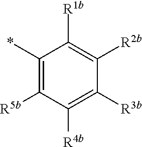

(one to five of R^{1b} to R^{5b} each represent a hydroxyalkyloxy group having 1-16 carbon atoms, and the remaining groups each represent a hydrogen atom, a halogen atom, an alkyl group having 1-4 carbon atoms, or an alkoxy group having 1-4 carbon atoms, or when R_{B} represents a structure of this formula, R^{5b} may form an ether bond (—O—) together with R^{5b} in formula (4). * represents a bonding site to R_{B}.)),
wherein a silazane compound is subjected to a reaction with a benzoyl compound of general formula (2):

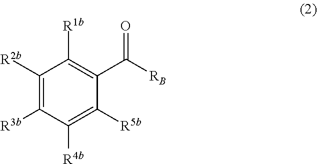

(in which one to five of R^{1b} to R^{5b} each represent a hydroxyalkyloxy group having 1-16 carbon atoms, the remaining groups each represent a hydrogen atom, a halogen atom, an alkyl group having 1-4 carbon atoms, or an alkoxy group having 1-4 carbon atoms, and
R_{B} represents a hydrogen atom, a hydroxy group, an alkoxy group having 1-6 carbon atoms, or a group of

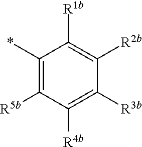

(one to five of R^{1b} to R^{5b} each represent a hydroxyalkyloxy group having 1-16 carbon atoms, and the remaining groups each represent a hydrogen atom, a halogen atom, an alkyl group having 1-4 carbon atoms, or an alkoxy group having 1-4 carbon atoms, or when R_{B} represents a structure of this formula, R^{5b} may form an ether bond (—O—) together with R^{5b} in formula (2). * represents a bonding site to R_{B}.)),
then causing the resulting product to react with an acid or a base to obtain a ketimine compound of general formula (3):

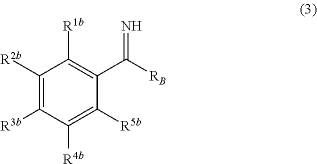

(in which one to five of R^{1b} to R^{5b} each represent a hydroxyalkyloxy group having 1-16 carbon atoms, the remaining groups each represent a hydrogen atom, a halogen atom, an alkyl group having 1-4 carbon atoms, or an alkoxy group having 1-4 carbon atoms, and
R_{B} represents a hydrogen atom, a hydroxy group, an alkoxy group having 1-6 carbon atoms, or a group of the following formula:

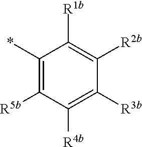

(one to five of R^{1b} to R^{5b} each represent a hydroxyalkyloxy group having 1-16 carbon atoms, and the remaining groups each represent a hydrogen atom, a halogen atom, an alkyl group having 1-4 carbon atoms, or an alkoxy group having 1-4 carbon atoms, or when R_{B} represents a structure of this formula, R^{5b} may form an ether bond (—O—) together with R^{5b} in formula (3). * represents a bonding site to R_{B}.)), and
causing the obtained ketimine compound to react with a reducing agent and an Fmoc-forming agent.

[3]A method for producing an alkylsilyloxy-substituted benzylamine compound of general formula (5):

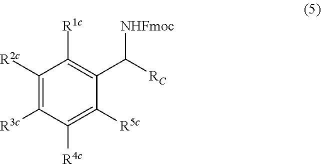

(in which one to five of R^{1c} to R^{5c} each represent an alkyloxy group having 1-29 carbon atoms and having 1-3 alkylsilyloxy groups as substituents, the remaining groups each represent a hydrogen atom, a halogen atom, an alkyloxy group having 1-4 carbon atoms, or an alkoxy group having 1-4 carbon atoms, and
R_{c} represents a hydrogen atom, a hydroxy group, an alkoxy group having 1-6 carbon atoms, or a group of the following formula:

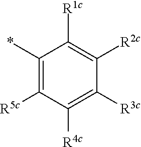

(one to five of R^{1c} to R^{5c} each represent an alkyloxy group having 1-29 carbon atoms and having 1-3 alkylsilyloxy groups as substituents, and the remaining groups each represent a hydrogen atom, a halogen atom, an alkyl group having 1-4 carbon atoms, or an alkoxy group having 1-4 carbon atoms, or when R_{c} represents a structure of this formula, R^{5c} may form an ether bond (—O—) together with R^{5c} in formula (5). * represents a bonding site to R_{C}.)),
wherein a silazane compound is subjected to a reaction with a benzoyl compound of general formula (2):

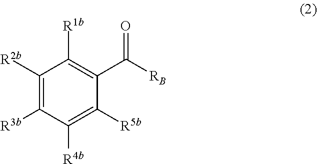

(in which one to five of R^{1b} to R^{5b} each represent a hydroxyalkyloxy group having 1-16 carbon atoms, the remaining groups each represent a hydrogen atom, a halogen atom, an alkyl group having 1-4 carbon atoms, or an alkoxy group having 1-4 carbon atoms, and
R_{B} represents a hydrogen atom, a hydroxy group, an alkoxy group having 1-6 carbon atoms, or a group of

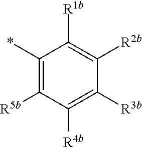

(one to five of R^{1b} to R^{5b} each represent a hydroxyalkyloxy group having 1-16 carbon atoms, and the remaining groups each represent a hydrogen atom, a halogen atom, an alkyl group having 1-4 carbon atoms, or an alkoxy group having 1-4 carbon atoms, or when R_{B} represents a structure of this formula, R^{5b} may form an ether bond (—O—) together with R^{5b} in formula (2). * represents a bonding site to R_{B}.)),
then causing the resulting product to react with an acid or a base to obtain a ketimine compound of general formula (3):

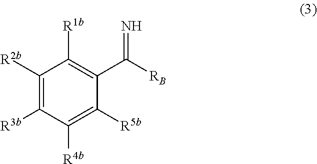

(in which one to five of R^{1b} to R^{5b} each represent a hydroxyalkyloxy group having 1-16 carbon atoms, the remaining groups each represent a hydrogen atom, a halogen atom, an alkyl group having 1-4 carbon atoms, or an alkoxy group having 1-4 carbon atoms, and
R_{B} represents a hydrogen atom, a hydroxy group, an alkoxy group having 1-6 carbon atoms, or a group of the following formula:

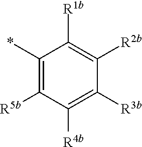

(one to five of R^{1b} to R^{5b} each represent a hydroxyalkyloxy group having 1-16 carbon atoms, and the remaining groups each represent a hydrogen atom, a halogen atom, an alkyl group having 1-4 carbon atoms, or an alkoxy group having 1-4 carbon atoms, or when R_{B} represents a structure of this formula, R^{5b} may form an ether bond (—O—) together with R^{5b} in formula (3) * represents a bonding site to R_{B}.)),
causing the obtained ketimine compound to react with a reducing agent and an Fmoc-forming agent to obtain a benzylamine compound of general formula (4):

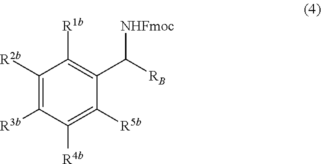

(in which one to five of R^{1b} to R^{5b} each represent a hydroxyalkyloxy group having 1-16 carbon atoms, the remaining groups each represent a hydrogen atom, a halogen atom, an alkyl group having 1-4 carbon atoms, or an alkoxy group having 1-4 carbon atoms, and
R_{B} represents a hydrogen atom, a hydroxy group, an alkoxy group having 1-6 carbon atoms, or a group of the following formula:

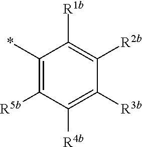

(one to five of R^{1b} to R^{5b} each represent a hydroxyalkyloxy group having 1-16 carbon atoms, and the remaining groups each represent a hydrogen atom, a halogen atom, an alkyl group having 1-4 carbon atoms, or an alkoxy group having 1-4 carbon atoms, or when R_{B} represents a structure of this formula, R^{5b} may form an ether bond (—O—) together with R^{5b} in formula (4). * represents a bonding site to R_{B}.)),
and then causing the benzylamine compound to react with an alkylsilylating agent.

[4] The production method according to any one of [1] to [3], wherein the alkylsilyloxy-substituted benzoyl compound of general formula (2) is obtained by causing a halogenated alcohol to react with a compound of general formula (1):

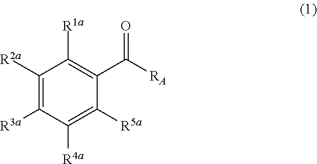

(in which one to five of R^{1a} to R^{5a} each represent a hydroxy group, the remaining groups each represent a hydrogen atom, a halogen atom, an alkyl group having 1-4 carbon atoms, or an alkoxy group having 1-4 carbon atoms, and
R_{A} represents a hydrogen atom, a hydroxy group, an alkoxy group having 1-6 carbon atoms, or a group of the following formula:

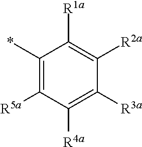

(one to five of R^{1a} to R^{5a} each represent a hydroxy group, and the remaining groups each represent a hydrogen atom, a halogen atom, an alkyl group having 1-4 carbon atoms, or an alkoxy group having 1-4 carbon atoms, or when R_{A} represents a structure of this formula, R^{5a} may form an ether bond (—O—) together with R^{5a} in formula (1) * represents a bonding site to R_{A}.)).

[5] A compound of general formula (6):

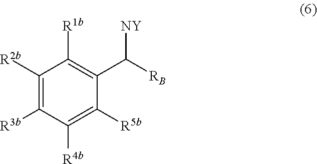

(in which one to five of R^{1b} to R^{5b} each represent a hydroxyalkyloxy group having 1-16 carbon atoms, the remaining groups each represent a hydrogen atom, a halogen atom, an alkyl group having 1-4 carbon atoms, or an alkoxy group having 1-4 carbon atoms, and
R_{B} represents a hydrogen atom, a hydroxy group, an alkoxy group having 1-6 carbon atoms, or a group of the following formula:

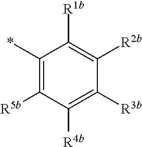

(one to five of R^{1b} to R^{5b} each represent a hydroxyalkyloxy group having 1-16 carbon atoms, and the remaining groups each represent a hydrogen atom, a halogen atom, an alkyl group having 1-4 carbon atoms, or an alkoxy group having 1-4 carbon atoms, or when R_{B} represents a structure of this formula, R^{5b} may form an ether bond (—O—) together with R^{5b} in formula (6), * represents a bonding site to R_{B}.), and
NY represents an imino group (=NH) or NHFmoc).

[6]A method for producing an alkylsilyloxy-substituted benzylamine compound of general formula (5):

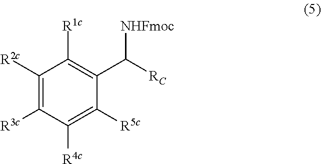

(in which one to five of R^{1c} to R^{5c} each represent an alkyloxy group having 1-29 carbon atoms and having 1-3 alkylsilyloxy groups as substituents, the remaining groups each represent a hydrogen atom, a halogen atom, an alkyloxy group having 1-4 carbon atoms, or an alkoxy group having 1-4 carbon atoms, and
R_{C} represents a hydrogen atom, a hydroxy group, an alkoxy group having 1-6 carbon atoms, or a group of the following formula:

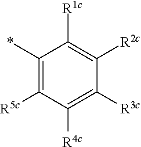

(one to five of R^{1c} to R^{5c} each represent an alkyloxy group having 1-29 carbon atoms and having 1-3 alkylsilyloxy groups as substituents, and the remaining groups each represent a hydrogen atom, a halogen atom, an alkyl group having 1-4 carbon atoms, or an alkoxy group having 1-4 carbon atoms, or when R_{C} represents a structure of this formula, R^{5c} may form an ether bond (—O—) together with R^{5c} in formula (5) * represents a bonding site to R_{C}.)),
wherein an alkylsilylating agent is subjected to a reaction with a benzylamine compound of general formula (4):

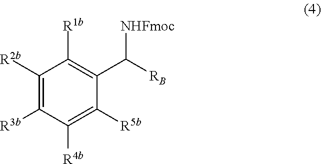

(in which one to five of R^{1b} to R^{5b} each represent a hydroxyalkyloxy group having 1-16 carbon atoms, the remaining groups each represent a hydrogen atom, a halogen atom, an alkyl group having 1-4 carbon atoms, or an alkoxy group having 1-4 carbon atoms, and
R_{B} represents a hydrogen atom, a hydroxy group, an alkoxy group having 1-6 carbon atoms, or a group of the following formula:

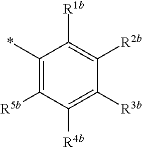

(one to five of R^{1b} to R^{5b} each represent a hydroxyalkyloxy group having 1-16 carbon atoms, and the remaining groups each represent a hydrogen atom, a halogen atom, an alkyl group having 1-4 carbon atoms, or an alkoxy group having 1-4 carbon atoms, or when R_{B} represents a structure of this formula, R^{5b} may form an ether bond (—O—) together with R^{5b} in formula (4), * represents a bonding site to R_{B}.)).

[7]A method for producing an alkylsilyloxy-substituted benzylamine compound of general formula (5):

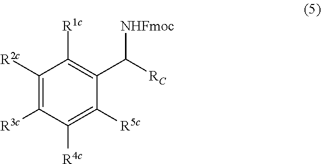

(in which one to five of R^{1c} to R^{5c} each represent an alkyloxy group having 1-29 carbon atoms and having 1-3 alkylsilyloxy groups as substituents, the remaining groups each represent a hydrogen atom, a halogen atom, an alkyloxy group having 1-4 carbon atoms, or an alkoxy group having 1-4 carbon atoms, and
R_{c} represents a hydrogen atom, a hydroxy group, an alkoxy group having 1-6 carbon atoms, or a group of the following formula:

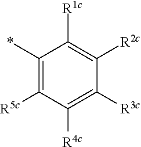

(one to five of R^{1c} to R^{5c} each represent an alkyloxy group having 1-29 carbon atoms and having 1-3 alkylsilyloxy groups as substituents, and the remaining groups each represent a hydrogen atom, a halogen atom, an alkyl group having 1-4 carbon atoms, or an alkoxy group having 1-4 carbon atoms, or when R_{C} represents a structure of this formula, R^{5c} may form an ether bond (—O—) together with R^{5c} in formula (5) * represents a bonding site to R_{C}.)),
wherein a reducing agent and an Fmoc-forming agent are subjected to a reaction with a ketimine compound of general formula (3):

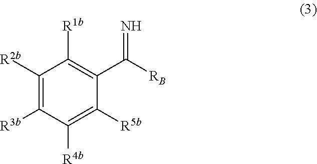

(in which one to five of R^{1b} to R^{5b} each represent a hydroxyalkyloxy group having 1-16 carbon atoms, the remaining groups each represent a hydrogen atom, a halogen atom, an alkyl group having 1-4 carbon atoms, or an alkoxy group having 1-4 carbon atoms, and
R_{B} represents a hydrogen atom, a hydroxy group, an alkoxy group having 1-6 carbon atoms, or a group of the following formula:

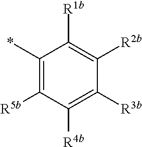

(one to five of R^{1b} to R^{5b} each represent a hydroxyalkyloxy group having 1-16 carbon atoms, and the remaining groups each represent a hydrogen atom, a halogen atom, an alkyl group having 1-4 carbon atoms, or an alkoxy group having 1-4 carbon atoms, or when R_{B} represents a structure of this formula, R^{5b} may form an ether bond (—O—) together with R^{5b} in formula (3). * represents a bonding site to R_{B}.)) to obtain a benzylamine compound of general formula (4):

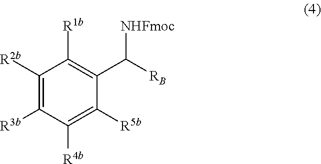

(in which one to five of R^{1b} to R^{5b} each represent a hydroxyalkyloxy group having 1-16 carbon atoms, the remaining groups each represent a hydrogen atom, a halogen atom, an alkyl group having 1-4 carbon atoms, or an alkoxy group having 1-4 carbon atoms, and
R_{B} represents a hydrogen atom, a hydroxy group, an alkoxy group having 1-6 carbon atoms, or a group of the following formula:

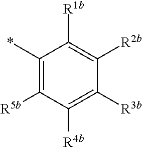

(one to five of R^{1b} to R^{5b} each represent a hydroxyalkyloxy group having 1-16 carbon atoms, and the remaining groups each represent a hydrogen atom, a halogen atom, an alkyl group having 1-4 carbon atoms, or an alkoxy group having 1-4 carbon atoms, or when R_{B} represents a structure of this formula, R^{5b} may form an ether bond (—O—) together with R^{5b} in formula (4). * represents a bonding site to R_{B}.)),
and then causing the benzylamine compound to react with an alkylsilylating agent.

### Advantageous Effects of Invention

According to the method of the present invention, it is not necessary to use a raw material having an issue of poor stability and difficulty in separation from an intermediate compound in the conventional method. In addition, since the intermediate compound is a solid, the raw material, a decomposition product of the raw material, and impurities generated or carried over in other systems can be easily removed by filtration or crystallization operation, for example. Further, the method does not pass through an alkylsilyloxy-substituted benzyl compound that is an acid-labile intermediate compound, and an alkylsilyloxy-substituted benzylamine compound can be obtained industrially advantageously.

### DESCRIPTION OF EMBODIMENTS

A reaction from a compound of general formula (1) to a compound of general formula (5) in the present invention is represented by the following reaction formula:

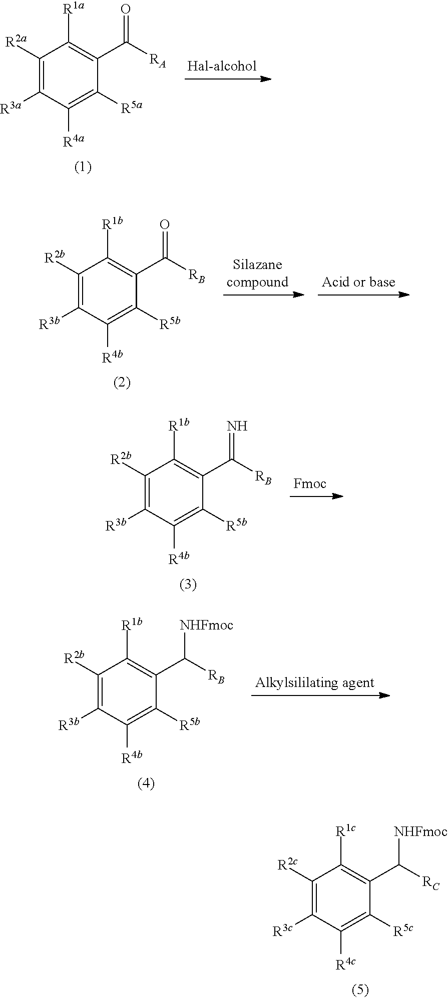

(in which one to five of R^{1a} to R^{5a} each represent a hydroxy group, the remaining groups each represent a hydrogen atom, a halogen atom, an alkyl group having 1-4 carbon atoms, or an alkoxy group having 1-4 carbon atoms, and
R_{A} represents a hydrogen atom, a hydroxy group, an alkoxy group having 1-6 carbon atoms, or a group of the following formula:

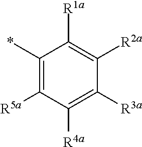

(one to five of R^{1a} to R^{5a} each represent a hydroxy group, and the remaining groups each represent a hydrogen atom, a halogen atom, an alkyl group having 1-4 carbon atoms, or an alkoxy group having 1-4 carbon atoms, or when R_{A} represents a structure of this formula, R^{5a} may form an ether bond (—O—) together with R^{5a} in formula (1). * represents a bonding site to R_{A}.),
one to five of R^{1b} to R^{5b} each represent a hydroxyalkyloxy group having 1-16 carbon atoms, the remaining groups each represent a hydrogen atom, a halogen atom, an alkyl group having 1-4 carbon atoms, or an alkoxy group having 1-4 carbon atoms, and
R_{B} represents a hydrogen atom, a hydroxy group, an alkoxy group having 1-6 carbon atoms, or a group of the following formula:

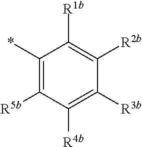

(one to five of R^{1b} to R^{5b} each represent a hydroxyalkyloxy group having 1-16 carbon atoms, and the remaining groups each represent a hydrogen atom, a halogen atom, an alkyl group having 1-4 carbon atoms, or an alkoxy group having 1-4 carbon atoms, or when R_{B} represents a structure of this formula, R^{5b} may form an ether bond (—O—) together with R^{5b} in formulas (2) to (4). * represents a bonding site to RE.),
one to five of R^{1c} to R^{5c} each represent an alkyloxy group having 1-29 carbon atoms and having 1-3 alkylsilyloxy groups as substituents, the remaining groups each represent a hydrogen atom, a halogen atom, an alkyl group having 1-4 carbon atoms, or an alkoxy group having 1-4 carbon atoms, and
R_{c} represents a hydrogen atom, a hydroxy group, an alkoxy group having 1-6 carbon atoms, or a group of the following formula:

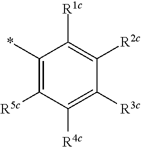

(one to five of R^{1c} to R^{5c} each represent an alkyloxy group having 1-29 carbon atoms and having 1-3 alkylsilyloxy groups as substituents, and the remaining groups each represent a hydrogen atom, a halogen atom, an alkyl group having 1-4 carbon atoms, or an alkoxy group having 1-4 carbon atoms, or when R_{c} represents a structure of this formula, R^{5c} may form an ether bond (—O—) together with R^{5a} in formula (5). * represents a bonding site to R_{C}.))

One aspect of the present invention is a method for producing a ketimine compound of general formula (3), in which a silazane compound is subjected to a reaction with the benzoyl compound of general formula (2), and then the resulting product is subjected to a reaction with an acid or a base.

Another aspect of the present invention is a method for producing a benzylamine compound of general formula (4), in which a silazane compound is subjected to a reaction with the benzoyl compound of general formula (2), then an acid or a base is subjected to a reaction with the resulting product to obtain a ketimine compound of general formula (3), and a reducing agent and an Fmoc-forming agent are subjected to a reaction with the ketimine compound.

Another aspect of the present invention is a method for producing an alkylsilyloxy-alkyloxybenzylamine compound of general formula (5), in which a silazane compound is subjected to a reaction with the benzoyl compound of general formula (2), then an acid or a base is subjected to a reaction with the resulting product to obtain a ketimine compound of general formula (3), a reducing agent and an Fmoc-forming agent are subjected to a reaction with the ketimine compound to obtain a benzylamine compound of general formula (4), and an alkylsilylating agent is subjected to a reaction with the obtained benzylamine compound. The term “alkylsilyloxy-substituted benzylamine compound” in the specification indicates this structure.

Here, the benzoyl compound of general formula (2) is preferably obtained by causing a halogenated alcohol to react with a hydroxybenzoyl compound of general formula (1).

Another aspect of the present invention is a method for producing an alkylsilyloxy-alkyloxybenzylamine compound of general formula (5), in which an alkylsilylating agent is subjected to a reaction with the benzylamine compound of general formula (4).

Another aspect of the present invention is a method for producing an alkylsilyloxy-alkyloxybenzylamine compound of general formula (5), in which a reducing agent and an Fmoc-forming agent are subjected to a reaction with the ketimine compound of general formula (3) to obtain a benzylamine compound of general formula (4), and an alkylsilylating agent is subjected to a reaction with the obtained benzylamine compound.

In addition, the ketimine compound of general formula (3) and the benzylamine compound of general formula (4) are novel compounds. Therefore, another aspect of the present invention provides a compound of the following general formula (6):

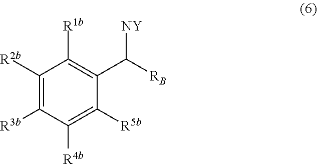

(in which one to five of R^{1b} to R^{5b} each represent a hydroxyalkyloxy group having 1-16 carbon atoms, the remaining groups each represent a hydrogen atom, a halogen atom, an alkyl group having 1-4 carbon atoms, or an alkoxy group having 1-4 carbon atoms, and
R_{B} represents a hydrogen atom, a hydroxy group, an alkoxy group having 1-6 carbon atoms, or a group of the following formula:

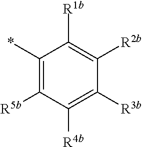

(one to five of R^{1b} to R^{5b} each represent a hydroxyalkyloxy group having 1-16 carbon atoms, and the remaining groups each represent a hydrogen atom, a halogen atom, an alkyl group having 1-4 carbon atoms, or an alkoxy group having 1-4 carbon atoms, or when R_{B} represents a structure of this formula, R^{5b} may form an ether bond (—O—) together with R^{5b} in formula (6) * represents a bonding site to R_{B}.), and
NY represents an imino group (=NH) or NHFmoc.)

First, the compound of general formula (1), which is a raw material compound, will be described.

One to five of R^{1a} to R^{5a} each represent a hydroxy group, and the remaining groups each represent a hydrogen atom, a halogen atom, an alkyl group having 1-4 carbon atoms, or an alkoxy group having 1-4 carbon atoms.

The number of hydroxy groups is preferably 1-4, more preferably 2 to 4, still more preferably 2 or 3, and further still more preferably 2.

Examples of the remaining groups include a hydrogen atom, a halogen atom, an alkyl group having 1-4 carbon atoms, and an alkoxy group having 1-4 carbon atoms, and a hydrogen atom, a halogen atom, or an alkyl group having 1-4 carbon atoms is preferable, and a hydrogen atom is more preferable.

Here, examples of the alkyl group having 1-4 carbon atoms include a methyl group, an ethyl group, a n-propyl group, an isopropyl group, and a n-butyl group. Examples of the alkoxy group having 1-4 carbon atoms include a methoxy group, an ethoxy group, a n-propyloxy group, an isopropyloxy group, and a n-butyloxy group. Examples of the halogen atom include a chlorine atom, a bromine atom, a fluorine atom, and an iodine atom.

R_{A} represents a hydrogen atom, a hydroxy group, an alkoxy group having 1-6 carbon atoms, or a group of the following formula:

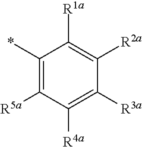

(one to five of R^{1a} to R^{5a} each represent a hydroxy group, and the remaining groups each represent a hydrogen atom, a halogen atom, an alkyl group having 1-4 carbon atoms, or an alkoxy group having 1-4 carbon atoms, or when R_{A} represents a structure of this formula, R^{5a} may form an ether bond (—O—) together with R^{5a} in formula (1). * represents a bonding site to R_{A}.)

Examples of the alkoxy group having 1-6 carbon atoms include a methoxy group, an ethoxy group, a n-propyloxy group, an isopropyloxy group, a n-butyloxy group, a tert-butyloxy group, an isobutyloxy group, a n-pentyl group, and a n-hexyl group. Among these groups, an alkoxy group having 1-5 carbon atoms is preferable, and an alkoxy group having 1-4 carbon atoms is more preferable.

The groups of R^{1a} to R^{5a} are preferably similar groups to those described above.

R_{A} is preferably a group of the following formula:

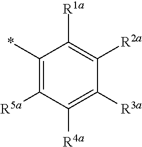

(one to five of R^{1a} to R^{5a} each represent a hydroxy group, and the remaining groups each represent a hydrogen atom, a halogen atom, an alkyl group having 1-4 carbon atoms, or an alkoxy group having 1-4 carbon atoms. * represents a bonding site to R_{A}.)

A halogenated alcohol (described as Hal-alcohol in the formula) is subjected to a reaction with the compound of general formula (1) to obtain a benzoyl compound of general formula (2).

Examples of the halogenated alcohol include a halogenated alcohol having 1-16 carbon atoms, and a halogenated alcohol having 2-16 carbon atoms is preferable, a halogenated alcohol having 4-16 carbon atoms is more preferable, a halogenated alcohol having 6-16 carbon atoms is still more preferable, and a halogenated alcohol having 8-16 carbon atoms is further still more preferable. Examples of the halogen atom include a bromine atom, a chlorine atom, an iodine atom, and a fluorine atom, and a bromine atom, a chlorine atom, and an iodine atom are preferable. Examples of the alcohol include a linear alcohol and a branched alcohol.

The reaction of the compound of general formula (1) and the halogenated alcohol is preferably performed in the presence of a base in a solvent.

Examples of the solvent used for the reaction include: an amide-based solvent such as dimethylformamide (hereinafter, referred to as DMF), diethylformamide, 1-methyl-2-pyrrolidone (hereinafter, referred to as NMP), or dimethylacetamide; a urea-based solvent such as 1,3-dimethyl-2-imidazolidinone (hereinafter, referred to as DMI); a halogenated solvent such as methylene chloride; an ether such as tetrahydrofuran or 2-methyltetrahydrofuran; a ketone such as acetone, methyl ethyl ketone, or methyl isobutyl ketone; an alcohol-based solvent such as methanol, ethanol, isopropanol, n-propanol, or n-butanol; a polar solvent such as acetonitrile; and a mixture thereof. Among these solvents, an amide-based solvent and a urea-based solvent are preferable, and DMF, DMI are more preferable.

Examples of the base include: an inorganic base such as lithium hydroxide, sodium hydroxide, potassium hydroxide, sodium carbonate, potassium carbonate, sodium hydrogen carbonate, potassium hydrogen carbonate, sodium hydride, or potassium hydride, and hydrates thereof; a metal alkoxide such as lithium methoxide, lithium ethoxide, sodium methoxide, sodium ethoxide, potassium methoxide, or potassium ethoxide; and an organic base such as 1,8-diazabicyclo [5.4.0]-7-undecene, diisopropylethylamine, triethylamine, dimethylaniline, or imidazole. Preferable examples of base include potassium carbonate and lithium hydroxide.

The reaction may be performed at a temperature of from 0° C. to 200° C., preferably from 50° C. to 150° C., and more preferably from 70° C. to 120° C. The reaction is preferably performed for from 15 minutes to 48 hours.

Since the benzoyl compound of general formula (2) obtained by this reaction can be isolated as a solid, this is easy to purify and its handleability is favorable. Isolation and purification may be easily performed by a conventional and industrially adoptable means, such as washing and recrystallization.

One to five of R^{1b} to R^{5b} in general formula (2) each represent a hydroxyalkyloxy group having 1-16 carbon atoms, and the remaining groups each represent a hydrogen atom, a halogen atom, an alkyl group having 1-4 carbon atoms, or an alkoxy group having 1-4 carbon atoms.

The number of hydroxyalkyloxy groups is preferably 1 to 4, more preferably 2 to 4, still more preferably 2 or 3, and further still more preferably 2.

Examples of the remaining groups include a hydrogen atom, a halogen atom, an alkyl group having 1-4 carbon atoms, and an alkoxy group having 1-4 carbon atoms, and a hydrogen atom, a halogen atom, or an alkyl group having 1-4 carbon atoms is preferable, and a hydrogen atom is more preferable.

R_{B} represents a hydrogen atom, a hydroxy group, an alkoxy group having 1-6 carbon atoms, or a group of the following formula:

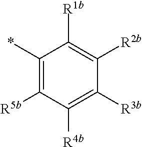

(one to five of R^{1b} to R^{5b} each represent a hydroxyalkyloxy group having 1-16 carbon atoms, and the remaining groups each represent a hydrogen atom, a halogen atom, an alkyl group having 1-4 carbon atoms, or an alkoxy group having 1-4 carbon atoms, or when R_{B} represents a structure of this formula, R^{5b} may form an ether bond (—O—) together with R^{5b} in formula (2). * represents a bonding site to R_{B}.)

Here, the group such as a hydroxyalkyloxy group having 1-16 carbon atoms is preferably a similar group to those of R^{1b} to R^{5b} described above.

Among the compounds of general formula (2), R_{B} is preferably a compound of the following formula:

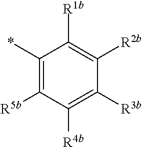

(one to five of R^{1b} to R^{4b} each represent a hydroxyalkyloxy group having 1-16 carbon atoms, and the remaining groups each represent a hydrogen atom, a halogen atom, an alkyl group having 1-4 carbon atoms, or an alkoxy group having 1-4 carbon atoms. * represents a bonding site to R_{B}.)

A silazane compound is subjected to a reaction with the benzoyl compound of general formula (2), and then the resulting product is subjected to a reaction with an acid or a base to obtain a ketimine compound of general formula (3). Specifically, when a silazane compound is subjected to a reaction with the compound of general formula (2) in the presence of a catalyst, a compound in which a trisubstituted silyl group is bonded to a hydroxyl group of the hydroxyalkyloxy group is obtained. Then, the obtained compound is subjected to a reaction with an acid or a base to deprotect the trisubstituted silyl group, thereby obtaining a ketimine compound of general formula (3).

The reaction using the silazane compound is a reaction of converting a carbonyl group in formula (2) into an imino group (=NH), and at the same time, a terminal alcohol of the hydroxyalkyloxy group of a side chain is protected with the trisubstituted silyl group.

The silazane compound is a compound having a Si—NH—Si bond. Examples of a chain silazane compound include a disubstituted disilazane (1,3-disubstituted disilazane), a tetrasubstituted disilazane (1,1,3,3-tetrasubstituted disilazane), and a hexasubstituted disilazane (1,1,1,3,3,3-hexasubstituted disilazane). A substituent in the silazane compound may be a linear, branched, or cyclic aliphatic group, and may be saturated or unsaturated. Among these groups, a saturated or unsaturated linear aliphatic group having 1-4 carbon atoms is preferable, tetramethyldisilazane (1,1,3,3-tetramethyldisilazane), hexamethyldisilazane (1,1,1,3,3,3-hexamethyldisilazane), 1,3-divinyl-1,1,3,3-tetramethyldisilazane, and 1,3-diphenyltetramethyldisilazane are preferable, and hexamethyldisilazane (1,1,1,3,3,3-hexamethyldisilazane) is most preferable.

Examples of the cyclic silazane compound include 2,2,4,4,6,6-hexasubstituted cyclotrisilazane. A substituent may be a linear, branched, or cyclic aliphatic group, and may be saturated or unsaturated. Among these groups, a saturated or unsaturated linear aliphatic group having 1-4 carbon atoms is preferable, and examples thereof include 2,2,4,4,6,6-hexamethylcyclotrisilazane and 2,4,6-trimethyl-2,4,6-trivinylcyclotrisilazane.

Specific examples of the catalyst include a catalyst containing a metal salt having Lewis acidity. Examples of the metal salt include a triflate salt, a nonaflate salt, and a trifluoromethanesulfonylimide salt, and specific examples thereof include Sc(OTf)₃, Y(OTf)₃, Sm(OTf)₃, Eu(OTf)₃, Gd(OTf)₃, Er(OTf)₃, Yb(OTf)₃, Fe(OTf)₃, In(OTf)₃, Sn(OTf)₃, Bi(OTf)₃, Sc(ONf)₃, and Sc(ONf)₃. Examples of the metal salt also include Sc(NO₃)₃ and BiBr₃, and also include tetrabutylammonium fluoride and tetrabutylammonium dihydrogenfluoride. The metal is particularly preferably at least one selected from the group consisting of Sc, Y, Eu, Er, Yb, Fe, Sn, and Bi, and the metal salt is particularly preferably Sc(OTf)₃.

The reaction can be performed in a solvent or without a solvent at from 0 to 150° C. for from 15 minutes to 48 hours. Examples of the solvent include: an aromatic hydrocarbon-based solvent such as chlorobenzene, toluene, or fluorobenzene; an ether-based solvent such as tetrahydrofuran or 1,4-dioxane; and a halogen-based solvent such as 1,2-dichloroethane, and chlorobenzene and toluene are particularly preferable, and toluene is most preferable. In addition, as a reaction accelerator, for example, water, alcohol, or silanol may be added.

After this reaction, an acid or a base is subjected to react with the resulting product to deprotect the terminal trisubstituted silyl group on the hydroxyalkyloxy group of the side chain, and the resulting product is converted into a terminal alcohol. Examples of the acid include an inorganic acid such as hydrochloric acid, trifluoroacetic acid, trifluoromethanesulfonic acid, 10-camphorsulfonic acid, or hydrogen fluoride-pyridine. Examples of the base include: an inorganic base such as lithium hydroxide, sodium hydroxide, potassium hydroxide, sodium carbonate, potassium carbonate, sodium hydrogen carbonate, potassium hydrogen carbonate, sodium hydride, or potassium hydride, and hydrates thereof; and a metal alkoxide such as lithium methoxide, lithium ethoxide, sodium methoxide, sodium ethoxide, potassium methoxide, or potassium ethoxide in the presence of tetraalkylammonium fluoride, cesium fluoride, potassium fluoride, or an alcohol solvent. Among these compounds, it is preferable to perform deprotection using a base, and in particular, it is desirable to cause an alkali metal carbonate or an alkali metal hydrogen carbonate to act in the presence of a C1-4 alcohol such as methanol or ethanol.

The deprotection reaction of the trisubstituted silyl group can be continuously performed by appropriately adding a solvent after completion of the reaction for converting the benzoyl group into an imino group (=NH). It is only required to perform the reaction at from 0 to 100° C. for from 15 minutes to 24 hours by adding a deprotecting agent for the trisubstituted silyl group.

Since the ketimine compound of general formula (3) obtained by this reaction can be isolated as a solid, this is easy to purify and its handleability is favorable. The isolation and purification are easily performed by a means that can be usually adopted industrially, such as washing and recrystallization.

A reducing agent and an Fmoc-forming agent are subjected to a reaction with the ketimine compound of general formula (3) to obtain a benzylamine compound of general formula (4)

This reaction is a reaction of converting an imino group (=NH) of formula (3) into a fluorenylmethyloxycarbonylamino group (—NHFmoc).

Examples of the reducing agent include a metal hydride such as lithium aluminum hydride, sodium borohydride, sodium cyanoborohydride, sodium triacetoxyborohydride, lithium borohydride, lithium triethylborohydride, lithium tri(sec-butyl) borohydride, sodium bis(2-ethoxyethoxy) aluminum hydride (hereinafter, referred to as SBAH), a borane complex, diisobutylaluminum hydride, or nickel borohydride. In addition, reduction using a silicon hydride such as a trialkylsilane or a reaction in which palladium-carbon acts as a catalyst in the presence of a hydrogen gas can also be mentioned. Among these compounds, lithium borohydride is most preferable.

The reduction reaction is preferably performed in an aromatic hydrocarbon-based solvent such as benzene or toluene, an ether-based solvent such as tetrahydrofuran, 2-methyltetrahydrofuran, or cyclopentyl methyl ether, an alcohol-based solvent such as methanol, ethanol, isopropanol, n-propanol, or n-butanol, or a mixture thereof at a temperature of from 0° C. to 100° C. for from 15 minutes to 48 hours.

Next, the Fmoc-forming agent is subjected to a reaction. The Fmoc-forming agent may be any compound as long as it can introduce a 9-fluorenylmethyloxycarbonyl (Fmoc) group, and for example, fluorenylmethyl halogenoformate, Fmoc-OSu, 9-fluorenylmethylcarbamate, 9-fluorenylmethylcarbazate, 1-(Fmoc-oxy) benzotriazole, and 9-fluorenylmethylpentafluorophenyl carbonate can be used. In addition, Fmoc-Amox described in Org.Process Res. Dev. 2017, 21 (10), 1533-41 can be used.

The Fmoc-forming reaction can be continuously performed after completion of the reduction reaction, and it is not necessary to change, for example, the solvent for the reaction. It is only required to add the Fmoc-forming agent and to perform the reaction at from 0 to 100° C. for from 10 minutes to three hours.

Since the benzylamine compound of general formula (4) obtained by this reaction can be isolated as a solid, this is easy to purify and its handleability is favorable. the isolation and purification are easily performed by a means that can be usually adopted industrially, such as washing and recrystallization.

By causing an alkylsilylating agent to react with the benzylamine compound of general formula (4), an alkylsilyloxy-alkyloxybenzylamine compound of general formula (5) can be obtained.

The alkylsilylating agent used in this reaction is a silylating agent having 1-3 alkylsilyls, and is preferably a silylating agent having an alkylsilyl group of any one of the following formulas (7) to (17). In the drawings, * represents a bonding point of a hydroxy group to an oxygen atom.

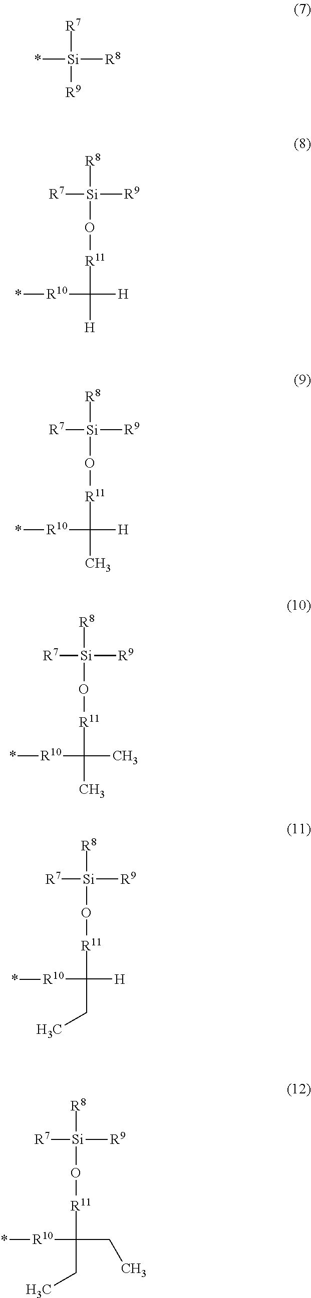

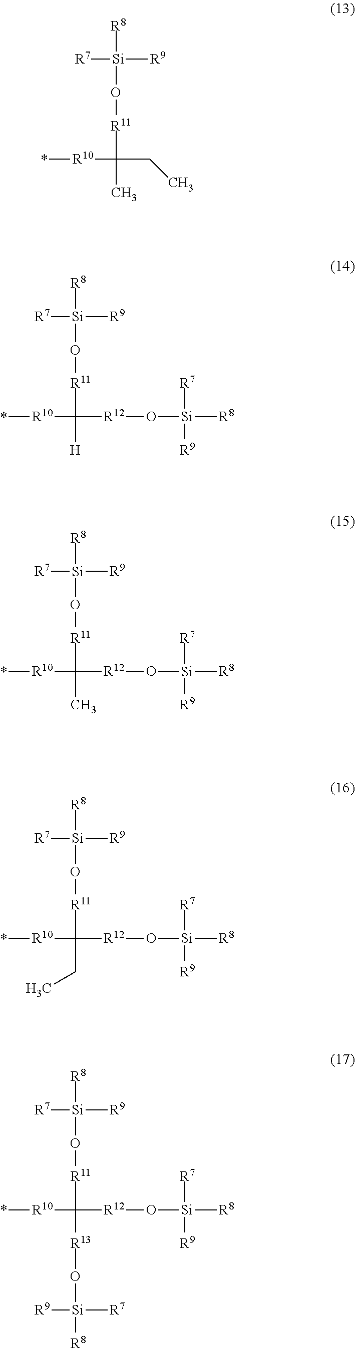

(in which R⁷, R⁸, and R⁹ are the same as or different from each other and each represent a linear or branched alkyl group having 1-6 carbon atoms or an aryl group optionally having a substituent, R¹⁰ represents a single bond or a linear or branched alkylene group having 1-3 carbon atoms, and R¹¹, R¹², and R¹³ each represent a linear or branched alkylene group having 1-3 carbon atoms.)

Here, examples of the linear or branched alkyl group having 1-6 carbon atoms include a methyl group, an ethyl group, an n-propyl group, an isopropyl group, an n-butyl group, an isobutyl group, a sec-butyl group, a tert-butyl group, a n-pentyl group, and a n-hexyl group. Among these groups, an alkyl group having 1-4 carbon atoms is more preferable, and a methyl group, a tert-butyl group, and an isopropyl group are still more preferable.

Examples of the aryl group optionally having a substituent include an aryl group having 6-10 carbon atoms, and specific examples thereof include a phenyl group and a naphthyl group which may have an alkyl group having 1-3 carbon atoms as a substituent. Among these groups, a phenyl group is more preferable.

Examples of the alkylsilylating agent include an alkylsilyl halide, an alkylsilyl imidazole, an alkylsilyl benztriazole, and an alkylsilyl trifluoromethanesulfonyl. Here, examples of the halogen atom include a bromine atom, a chlorine atom, and an iodine atom.

The reaction of the benzylamine compound of general formula (4) and the alkylsilylating agent is preferably performed in the presence of a base in a solvent.

Examples of the solvent used for the reaction include: an amide-based solvent such as DMF, diethylformamide, NMP, or dimethylacetamide; a urea-based solvent such as DMI; a halogenated solvent such as methylene chloride; an ether such as tetrahydrofuran or 2-methyltetrahydrofuran; a ketone such as acetone, methyl ethyl ketone, or methyl isobutyl ketone; a polar solvent such as acetonitrile; and a mixture thereof. Among these solvents, an amide-based solvent and a urea-based solvent are preferable, and DMF, NMP, and DMI are more preferable.

Examples of the base include: an inorganic salt such as lithium hydroxide, sodium hydroxide, potassium hydroxide, sodium carbonate, potassium carbonate, sodium hydrogen carbonate, potassium hydrogen carbonate, sodium hydride, or potassium hydride, and hydrates thereof; a metal alkoxide such as lithium methoxide, lithium ethoxide, sodium methoxide, sodium ethoxide, potassium methoxide, or potassium ethoxide; and an organic base such as 1,8-diazabicyclo [5.4.0]-7-undecene, diisopropylethylamine, triethylamine, dimethylaniline, or imidazole. Preferable examples of the base include imidazole.

The reaction may be performed at a temperature of from 0° C. to 150° C., preferably from 20 to 100° C., and more preferably from 30 to 50° C. The reaction is preferably performed for from 15 minutes to 48 hours.

In this reaction, a trace amount of silyl-based compound derived from the alkylsilylating agent may be produced as a by-product in the reaction mixture. In that case, the silyl-based compound is preferably removed by liquid-liquid separation. The alkylsilyloxy-substituted benzylamine compound (5) as a target product is dissolved in an alkane-based solvent such as heptane. Meanwhile, it is preferable to perform liquid-liquid separation using a polar solvent such as acetonitrile, methanol, DMF, or dimethyl sulfoxide. For the polar solvent, a solvent mixture with water is preferably used, and in particular, a combination of acetonitrile and water is preferable.

One to five of R^{1c} to R^{5c} in general formula (5) each represent an alkyloxy group having 1-29 carbon atoms and having 1-3 alkylsilyloxy groups as substituents, and the remaining groups each represent a hydrogen atom, a halogen atom, an alkyl group having 1-4 carbon atoms, or an alkoxy group having 1-4 carbon atoms. A structure of the alkyloxy group having 1-29 carbon atoms and having alkylsilyloxy groups as substituents is represented by the following formula:

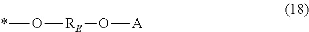

(R_{B} represents a linear or branched alkyl group having 1-16 carbon atoms, A represents any one of formulas (7) to (17). * represents a bonding site to a carbon atom on a benzoyl skeleton, a diphenyl ketone skeleton, or a xanthone skeleton which is a core.)

The number 1-29 of carbon atoms of the alkyloxy group refers to a total value of the number of carbon atoms of a hydroxyalkyloxy chain having 1-16 carbon atoms derived from a halogenated alcohol and the number of carbon atoms contained in R¹⁰, R¹¹, R¹², and R¹³ in the silylating agent of any one of formulas (7) to (17).

The alkyloxy group having 1-29 carbon atoms and having 1-3 alkylsilyloxy groups as substituents is preferably 1 to 4, more preferably 2 to 4, still more preferably 2 or 3, and further still more preferably 2.

Examples of the remaining groups include a hydrogen atom, a halogen atom, an alkyl group having 1-4 carbon atoms, and an alkoxy group having 1-4 carbon atoms, and a hydrogen atom, a halogen atom, or an alkyl group having 1-4 carbon atoms is preferable, and a hydrogen atom is more preferable.

R_{c} represents a hydrogen atom, a hydroxy group, an alkoxy group having 1-6 carbon atoms, or a group of the following formula:

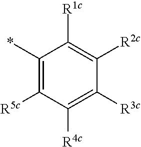

(one to five of R^{1b} to R^{5b} each represent an alkyloxy group having 1-29 carbon atoms and having 1-3 alkylsilyloxy groups as substituents, and the remaining groups each represent a hydrogen atom, a halogen atom, an alkyl group having 1-4 carbon atoms, or an alkoxy group having 1-4 carbon atoms, or when R_{C} represents a structure of this formula, R^{5c} may form an ether bond (—O—) together with R^{5c} in formula (5). * represents a bonding site to R_{C}.)

Here, examples of the alkyloxy group having 1-29 carbon atoms and having 1-3 alkylsilyloxy groups as substituents include the same groups as the alkylsilyl group. In addition, other substituents of R^{1c} to R^{5c} are preferably similar to those of R^{1b} to R^{5b} described above.

By the alkylsilyloxylation reaction, the alkylsilyloxy-alkyloxybenzylamine compound of general formula (5) is obtained.

Since the alkylsilyloxy-alkyloxybenzylamine compound of general formula (5) obtained by this reaction can be isolated as a solid, this is easy to purify and its handleability is favorable. The isolation and purification are easily performed by a means that can be usually adopted industrially, such as washing and recrystallization. The alkylsilyloxy-alkyloxybenzylamine compound of general formula (5) is useful as a liquid phase peptide synthesis carrier as described in Patent Literatures 1 to 3.

According to the method of the present invention, each of the benzoyl compound of general formula (2), the ketimine compound of general formula (3), the benzylamine compound of general formula (4), and the alkylsilyloxy-alkyloxybenzylamine compound of general formula (5) can be obtained as a solid. In the present invention, the term “solid” encompasses a solid having a crystal structure and an amorphous-like solid. When each compound has the following structure, the compound is easily obtained as a solid having a form closer to a crystal structure, and easily exhibits a beneficial function as a liquid phase peptide synthesis carriers (Tag).

In the compound of general formula (1), preferably, one to five of R^{1a} to R^{5a} are each a hydroxy group, and the remaining groups are each a hydrogen atom. Particularly preferably, R^{3a} is a hydroxy group, and the remaining groups are each a hydrogen atom. R_{A} is preferably a group of the following formula:

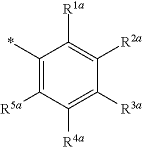

(Preferably, one to five of R^{1a} to R^{5a} are each a hydroxy group, and the remaining groups are each a hydrogen atom. Particularly preferably, R^{3a} is a hydroxy group, and the remaining groups are each a hydrogen atom (* represents a bonding site to R_{A}.)).

In the compound of general formula (2), (3), or (4), preferably, one to five of R^{1b} to R^{5b} are each a hydroxyalkyloxy group having 1-16 carbon atoms, and the remaining groups are each a hydrogen atom. Particularly preferably, R^{3a} is a hydroxyalkyloxy group having 1-16 carbon atoms, and the remaining groups are each a hydrogen atom. The number of carbon atoms of the hydroxyalkyloxy group is more preferably 4 to 16, still more preferably 8 to 16, further still more preferably 10 to 16, and most preferably 11 to 16. R_{B} is preferably a group of the following formula:

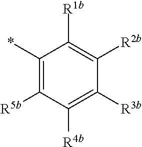

(Preferably, one to five of R^{1b} to R^{5b} are each a hydroxyalkyloxy group having 1-16 carbon atoms, and the remaining groups are each a hydrogen atom. Particularly preferably, R^{3b} is a hydroxyalkyloxy group having 1-16 carbon atoms, and the remaining groups are each a hydrogen atom. The number of carbon atoms of the hydroxyalkyloxy group is more preferably 4 to 16, still more preferably 8 to 16, further still more preferably 10 to 16, and most preferably 11 to 16. * represents a bonding site to R_{B}.)

In the compound of general formula (5), preferably, one to five of R^{1c} to R^{5c} are each an alkyloxy group having 1-29 carbon atoms and having 1-3 alkylsilyloxy groups as substituents, and the remaining groups are each a hydrogen atom. More preferably, R^{3b} is an alkylsilyloxy group having 1-29 carbon atoms and having one alkylsilyloxy group as a substituent, and the remaining groups are each a hydrogen atom. Particularly, the alkylsilyloxy group is preferably synthesized with an alkylsilylating agent of formula (7). In formula (7), R⁷, R⁸, and R⁹ are each preferably a linear or branched alkyl group having 1-6 carbon atoms or a phenyl group.

R_{c} is preferably a group of the following formula:

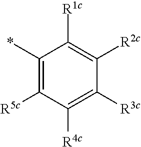

(Preferably, one to five of R^{1c} to R^{5c} are each an alkyloxy group having 1-29 carbon atoms and having 1-3 alkylsilyloxy groups as substituents, and the remaining groups are each a hydrogen atom. More preferably, R^{3b} is an alkylsilyloxy group having 1-29 carbon atoms and having one alkylsilyloxy group as a substituent, and the remaining groups are each a hydrogen atom. The alkylsilyloxy group is particularly preferably synthesized with the alkylsilylating agent of formula (7). In formula (7), R⁷, R⁸, and R⁹ are each preferably a linear or branched alkyl group having 1-6 carbon atoms or a phenyl group. * represents a bonding site to R_{C}.)

### EXAMPLES

Hereinafter, the present invention will be described in detail with reference to Example. However, the present invention is not limited to the Example.

### Comparative Example 1: Synthesis of Fmoc-D2-STag by Conventional Method

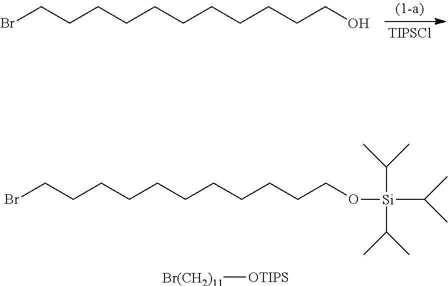

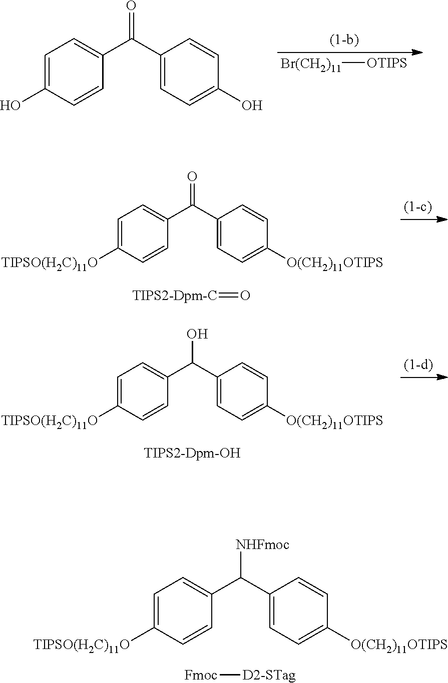

(Hereinafter, Br—(CH₂)₁₁-OTIPS, TIPS2-Dpm-C=O, TIPS2-Dpm-OH, and Fmoc-D2-STag represent structures in the formula.)

(1-a) Br—(CH₂)₁₁—O-TIPS

In 12.8 mL of dichloromethane, 0.90 g (3.58 mmol) of 11 bromo-1-undecanol was dissolved, 0.61 g (8.96 mmol) of imidazole was added thereto, the resulting mixture was cooled to 5° C., and 0.91 mL (4.30 mmol) of triisopropylsilyl chloride (hereinafter, referred to as TIPS-Cl) was added dropwise thereto. After five minutes, the temperature was returned to room temperature, and the mixture was stirred for two hours. To the reaction solution, 51.2 mL of cyclopentyl methyl ether was added, and the resulting mixture was washed once with 12.8 mL of distilled water, once with 12.8 mL of a 1M hydrochloric acid aqueous solution, and three times with 12.8 mL of distilled water, and the organic layer was distilled off. The residue was dissolved in 51.2 mL of heptane, and the resulting solution was liquid-separated and washed with 25.6 mL of acetonitrile. To the obtained heptane layer, 12.8 mL of heptane was added, and the resulting layer was liquid-separated and washed with 25.6 mL of acetonitrile. The liquid separation and washing with heptane and acetonitrile was further performed once, and then a solvent was distilled off to obtain 1.45 g (yield 99.3%) of Br—(CH₂)₁₁—O-TIPS. The obtained Br—(CH₂)₁₁—O-TIPS was oily.

¹H-NMR (400 MHz, CDCl₃) δ 1.03-1.20 (m, 21H), 1.24-1.49 (m, 14H), 1.54 (quin., 2H), 1.85 (quin., 2H), 3.41 (t, 2H), 3.66 (t, 2H)

ESIMS MH+407.1

### (1-b) TIPS2-Dpm-C=O

In 3.2 mL of DMF, 9.81 g (24.1 mmol) of Br—(CH₂)₁₁-OTIPS, 2.29 g (10.7 mmol) of 4,4′-dihydroxybenzophenone, and 5.33 g (38.5 mmol) of potassium carbonate were suspended, and the resulting suspension was heated to 85° C. and stirred for two hours. The reaction solution was filtered, and the filtered product was washed with 150 mL of heptane. The filtrate was liquid-separated. To the obtained heptane layer, 71 mL of heptane was added, and the resulting layer was liquid-separated and washed with 71 mL of DMF. The liquid separation and washing with heptane and DMF were performed once more. To the obtained heptane layer, 71 mL of heptane was added, and the resulting layer was liquid-separated and washed once with 71 mL of a 1M hydrochloric acid aqueous solution, once with 71 mL of a 5% sodium hydrogen carbonate aqueous solution, and once with 71 mL of distilled water. To the obtained heptane layer, 71 mL of heptane was added, and the resulting layer was liquid-separated and washed once with 71 mL of DMF and one with 71 mL of acetonitrile. The heptane layer was concentrated under reduced pressure to obtain 10.7 g of TIPS2-Dpm-C=O. The obtained TIPS2-Dpm-C=O was in a form of viscous oil.

¹H-NMR (400 MHz, CDCl₃) δ 1.04-1.08 (m, 42H), 1.20-1.39 (m, 24H), 1.41-1.49 (m, 4H), 1.49-1.57 (m, 4H), 1.71-1.85 (m, 4H), 3.67 (t, 4H), 4.03 (t, 4H), 6.94 (d, 4H), 7.77 (d, 4H)

¹³C-NMR (100 MHz, CDCl₃) δ 12.2 (6C), 18.2 (12C), 26.0 (2C), 26.2 (2C), 29.2-29.8 (12C), 33.2 (2C), 63.7 (2C), 68.4 (2C), 114.0 (4C), 130.7 (2C), 132.4 (4C), 162.6 (2C), 194.6

ESIMS MNa+889.8

### (1-c) TIPS2-Dpm-OH

In a solution mixture of 7.1 mL of THF (anhydrous) and 0.36 mL of methanol, 0.81 g (0.93 mmol) of TIPS2-Dpm-C=O was dissolved, 42 mg (1.12 mmol) of sodium borohydride was added thereto, and the resulting mixture was stirred for 1.5 hours. To the reaction solution, 0.89 mL of a 1M hydrochloric acid aqueous solution was added to stop the reaction, 20.3 mL of cyclopentyl methyl ether was added thereto, the resulting mixture was washed once with 6.1 mL of a 1M aqueous hydrochloric acid aqueous solution, once with 6.1 mL of a 5% sodium hydrogen carbonate aqueous solution, and once with 6.1 mL of distilled water, and the organic layer was concentrated under reduced pressure. The obtained residue was dissolved in 20.0 mL of heptane, and the resulting solution was liquid-separated and washed with 10.0 mL of DMF. To the obtained heptane layer, 10.0 mL of heptane was added, and the resulting layer was liquid-separated and washed with 10.0 mL of acetonitrile. The liquid separation and washing with heptane and acetonitrile was further performed once, and then the heptane layer was concentrated under reduced pressure to obtain 0.81 g of TIPS2-Dpm-OH.

The obtained TIPS2-Dpm-OH was in a form of viscous oil.

¹H-NMR (400 MHz, Benzene-d) δ 1.12-1.16 (m, 42H), 1.23-1.54 (m, 32H), 1.57-1.71 (m, 4H), 1.79 (s, 1H), 3.68 (t, 8H), 5.61 (s, 1H), 6.84-6.89 (m, 4H), 7.27-7.33 (m, 4H)

¹³C-NMR (100 MHz, Benzene-d) δ 12.8 (6C), 18.7 (12C), 26.7 (2C), 26.8 (2C), 30.2-30.5 (12C), 33.9 (2C), 64.1 (2C), 68.3 (2C), 75.9, 114.9 (4C), 128.6 (4 C), 137.8 (2C), 159.4 (2C)

A ratio of HO—(CH₂)₁₁-OTIPS contained as impurities in the crude purified product of TIPS2-Dpm-OH was analyzed by HPLC, and 5.1% by weight of HO—(CH₂)₁₁-OTIPS was contained.

### Analysis Conditions (HPLC)

Column: YMC-Triart C18

(Inner diameter: 3.0 mm, length: 100 mm, and particle diameter: 1.9 μm)

Mobile phase A: a solution containing 0.01M ammonium formate and having a ratio of acetonitrile:water=8:2

Mobile phase B: a solution containing 0.01M ammonium formate and having a ratio of isopropanol:water=100:1

Flow rate: 0.25 mL/min

Column temperature: 35° C.

Detection wavelength: 200 nm

Gradient conditions: 0% B (0 min)->0% B (5 min)->100% B (22 min)->100% B (27 min)->0% B (29 min)->0% B (31 min)

### (1-d) Fmoc-D2-STag

In 6.5 mL of toluene, 1.00 g (1.15 mmol) of TIPS2-Dpm-OH and 0.29 g (1.25 mmol) of Fmoc-NH2 were dissolved. To the resulting solution, 0.04 g (0.32 mmol) of oxalic acid dihydrate was added, and then the resulting mixture was heated to 85° C. and stirred for four hours. To the resulting solution, 10 mL of heptane and 10 mL of 90% methanol water were added, and the resulting mixture was liquid-separated and washed. To the obtained upper layer, 5 mL of 5% sodium bicarbonate water was added, and the resulting mixture was liquid-separated and washed. An operation of adding 10 mL of 90% methanol water to the obtained upper layer and liquid-separating and washing the resulting layer was performed three times. The obtained upper layer was concentrated under reduced pressure. The obtained residue was dissolved in 2 mL of tetrahydrofuran, the tetrahydrofuran solution was added dropwise to 20 mL of stirred methanol, and the resulting mixture was stirred for 30 minutes after the dropwise addition. The process liquid in which a target product was crystallized was filtered, and the filtered product was washed with methanol. The washed product was dried under reduced pressure to obtain 0.94 g (yield: 74.9%) of Fmoc-D2-STag. The obtained Fmoc-D2-STag was a solid.

¹H-NMR (400 MHz, CDCl3) δ 0.99-1.12 (m, 6H), 1.06 (s, 36H), 1.18-1.38 (m, 24H), 1.38-1.48 (m, 4H), 1.48-1.60 (m, 4H), 1.70-1.83 (m, 4H), 3.66 (dd, 4H), 3.93 (dd, 4H), 4.21 (br, 1H), 4.44 (d, 1H), 50.00-5.13 (br, 0.18H), 5.20-5.32 (br d, 0.82H), 5.70-5.80 (br, 0.18H), 5.82-5.92 (br d, 0.82H), 6.84 (d, 4H), 7.11 (d, 4H), 7.28-7.34 (m, 2H), 7.34-7.47 (m, 2H), 7.55-7.65 (br d, 2H), 7.70-7.85 (br d, 2H)

¹³C-NMR (100 MHz, CDCl3) δ 12.0, 18.0, 25.8, 26.0, 29.3, 29.40, 29.55 (4C), 29.62, 33.0, 63.5, 680.0, 77.2, 114.5, 119.9, 125.0, 127.0, 127.6, 128.3, 133.8, 141.3, 143 0.9, 155.5, 158.4

ESIMS MNa+1112.8

A ratio of HO—(CH₂)₁₁-OTIPS contained as impurities in the crude purified product of Fmoc-D2-STag was analyzed by HPLC under the same conditions as in step (1-c), and HO—(CH₂)₁₁-OTIPS contained as impurities in the crude purified product of TIPS2-Dpm-OH was carried over as it was. There was also an issue that TIPS2-Dpm-OH as a target product in step (1-c) was decomposed by a trace amount of acid and dimerized during storage. For example, as TIPS2-Dpm-OH was stored at 30° C., 30 days later a dimer was generated at a ratio of 10.0%. Therefore, it was difficult to store TIPS2-Dpm-OH for a long period of time. In addition, in the step (1-d), a side reaction in which a TIPS group was deprotected by an acid catalyst also occurred, and in Comparative Example, a dealkylsilyl body was generated at a ratio of 0.2%. Both side reactions reduced a yield of Fmoc-D2-STag.

### Analysis Conditions of Fmoc-D2-STag, Dimer and Dealkylsilyl Body of TIPS2-Dpm-OH (HPLC)

Column: YMC-Triart C18

(Inner diameter: 3.0 mm, length: 100 mm, and particle diameter: 1.9 μm)

Mobile phase A: obtained by adding 0.1% v/v formic acid to a solution having a ratio of isopropanol acetonitrile:water=5:90:

Mobile phase B: obtained by adding 0.1% v/v formic acid to isopropanol

Flow rate: 0.35 mL/min

Column temperature: 35° C.

Detection wavelength: 254 nm

Gradient conditions: 40% B (0 min)->40% B (3 min)->100% B (28 min)->100% B (40 min)->40% B (41 min)->40% B (51 min)

### Example 1: Synthesis of Fmoc-D2-STag by Method of Present Invention

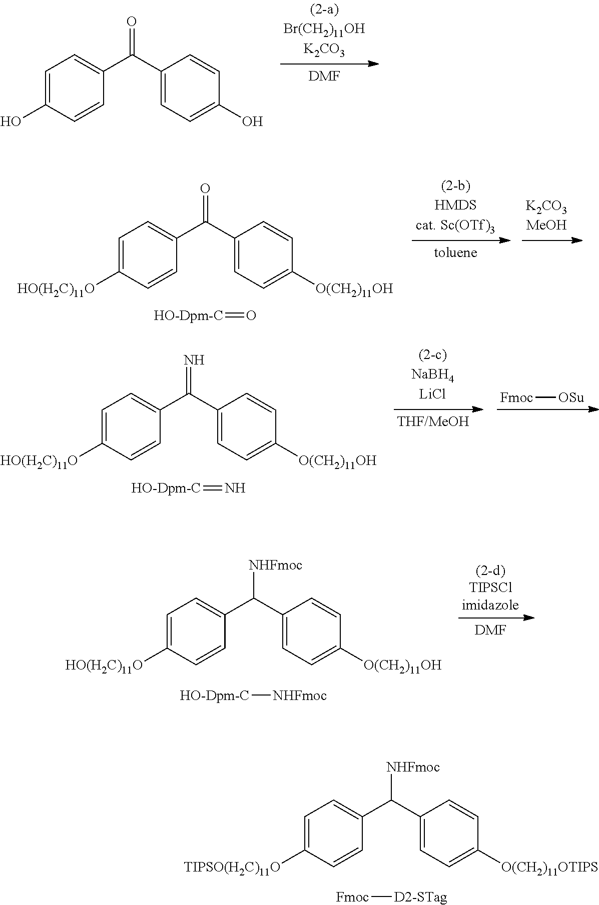

(Hereinafter, HO-Dpm-C=O, HO-Dpm-C=NH, HO-Dpm-C-NHFmoc, and Fmoc-D2-STag represent structures in the formula.)

### (2-a) HO-Dpm-C=O

In 1.75 L of DMF, 484 g (1.93 mol) of Br—(CH₂)₁₁—OH, 187 g (875 mmol) of 4.4′-dihydroxybenzophenone, and 339 g (2.45 mol) of potassium carbonate were suspended, and the resulting suspension was heated to 90° C. and stirred for four hours. The reaction solution was adjusted to 80° C., 2.25 L of water was added thereto, and the resulting mixture was stirred at the same temperature for one hour. The slurry was cooled to 30° C. and then filtered. The filtered product was washed once with 0.56 L of water and once again with 0.56 L of water. After washing, the filtered product was collected. To the filtered product, 2.25 L of water was added, and the resulting mixture was stirred at 80° C. for one hour. The slurry was cooled to 30° C. and then filtered. The filtered product was washed once with 0.56 L of water and once again with 0.56 L of water. After washing, the filtered product was collected. To the filtered product, 1.78 L of methanol was added, and the resulting mixture was stirred at 60° C. for one hour. The slurry was cooled to 30° C. and then filtered. The filtered product was washed once with 0.56 L of methanol and once again with 0.56 L of methanol. Thereafter, the washed product was dried under reduced pressure at 40° C. to obtain 474 g (yield: 97.6%) of HO-Dpm-C=O. The obtained HO-Dpm-C=O was a solid.

¹H-NMR (400 MHz, Pyridine-D5) δ 1.20-1.39 (m, 20H), 1.39-1.59 (m, 8H), 1.70-1.84 (m, 8H), 3.90 (t, J=6.2 Hz, 4H), 4.04 (t, J=6.6 Hz, 4H), 5.93 (brs, 2 H), 7.17 (d, J=8.7 Hz, 4H), 8.04 (d, J=8.7 Hz, 4H)

¹³C-NMR (100 MHz, Pyridine-D5) δ 26.3, 26.6, 29.4, 29.6, 29.8, 29.9, 30.0, 33.8, 62.1, 68.5, 114.6, 1310.1, 132.6, 163.0, 194.0

ESIMS MNa+577.4

### (2-b) HO-Dpm-C=NH

In 0.83 L of toluene, 461 g (831 mmol) of HO-Dpm-C=O and 40.9 g (83.1 mmol) of scandium trifluoromethanesulfonate were suspended, 0.70 L of hexamethyldisilazane was added thereto, and the resulting mixture was heated to 90° C. and stirred for 21 hours. The reaction solution was cooled to 30° C., 0.83 L of methanol and 345 g (2.49 mol) of potassium carbonate were added thereto, and the resulting mixture was stirred at 30° C. for three hours. To the reaction solution, 1.84 L of water was added, and the resulting mixture was stirred at 50° C. for one hour. The slurry was cooled to 30° C. and then filtered. The filtered product was washed once with 0.92 L of water and once again with 0.92 L of water. After washing, the filtered product was collected. To the filtered product, 1.84 L of 50% methanol water was added, and the resulting mixture was stirred at 50° C. for one hour. The slurry was cooled to 30° C. and then filtered. The filtered product was washed once with 0.92 L of 50% methanol water and once again with 0.92 L of 50% methanol water. After washing, the filtered product was collected. To the filtered product, 1.84 L of acetonitrile was added, and the resulting mixture was stirred at 50° C. for one hour. The slurry was cooled to 30° C. and then filtered. The filtered product was washed once with 1.84 L of acetonitrile. Thereafter, the washed product was dried under reduced pressure at 40° C. to obtain 437 g (yield: 94.9%) of HO-Dpm-C=NH. The obtained HO-Dpm-C=NH was a solid.

¹H-NMR (400 MHz, Pyridine-D5) δ 1.18-1.37 (m, 20H), 1.37-1.45 (m, 4H), 1.45-1.55 (m, 4H), 1.70-1.82 (m, 4H), 3.88 (dd, 4H), 3.99 (dd, 4H), 5.93 (s, 4H), 7.12 (d, 4H), 7.60-8.20 (br, 4H), 10.33 (br s, 1H)

¹³C-NMR (100 MHz, Pyridine-D5) δ 26.3, 26.5, 29.5, 29.6, 29.8 (2C), 29.9, 30.0, 33.8, 62.1, 68.3, 114.6, 130.7 (br, 2C), 161.3, 176.2

ESIMS MNa+576.4

### (2-c) HO-Dpm-C-NHFmoc

In 1.25 L of tetrahydrofuran, 104.65 g (2.47 mol) of lithium chloride and 93.40 g (2.47 mmol) of sodium borohydride were suspended, and the resulting suspension was stirred at 10° C. or lower for ten minutes. To the suspension, 3.74 L of methanol and 414 g (748 mmol) of HO-Dpm-C=NH were added, and the resulting mixture was heated to 40° C. and stirred for two hours. To the mixture, 328 g (973 mmol) of Fmoc-OSu was added, and the resulting mixture was stirred at 40° C. for two hours. To the mixture, 4.97 L of 50% methanol water was added, and the resulting mixture was stirred at 40° C. for one hour. The slurry was cooled to 30° C. and then filtered. The filtered product was washed once with 2.9 L of 50% methanol water. After washing, the filtered product was collected. To the filtered product, 4.97 L of 50% methanol water was added, and the resulting mixture was stirred at 40° C. for one hour. The slurry was cooled to 30° C. and then filtered. The filtered product was washed once with 2.90 L of 50% methanol water and once again with 2.90 L of 50% methanol water. After washing, the filtered product was collected. To the filtered product, 4.97 L of acetonitrile was added, and the resulting mixture was stirred at 40° C. for one hour. The slurry was cooled to 30° C. and then filtered. The filtered product was washed once with 2.90 L of acetonitrile. Thereafter, the washed product was dried under reduced pressure at 40° C. to obtain 549 g (yield: 94.3%) of HO-Dpm-C-NHFmoc. The obtained HO-Dpm-C-NHFmoc was a solid.

¹H-NMR (400 MHz, CDCl3) δ 1.20-1.39 (m, 24H), 1.38-1.50 (m, 4H), 1.50-1.60 (m, 4H), 1.67-1.84 (m, 4H), 3.63 (ddd, 4H), 3.93 (dd, 4H), 4.21 (t, 1H), 4.44 (d, 1H), 50.00-5.20 (br, 0.18H), 5.33 (d, 0.82H), 5.62-5.70 (br, 0.18H), 5.71 (d, 0.82H), 6.84 (d, 4H), 7.11 (d, 4H), 7.27-7.34 (m, 2H), 7.34-7.46 (m, 2H), 7.49-7.65 (br d, 2H), 7.66-7.84 (br d, 2H)

¹³C-NMR (100 MHz, CDCl3) δ 25.7, 26.0, 29.2, 29.3, 29.37, 29.45 (2C), 29.49 (2C), 29.53, 32.8, 630.1, 68.0, 77.2, 114.5, 119.9, 125.0, 127.0, 127.6, 128.3, 133.8, 141.3, 143.9, 155.5, 158.4

ESIMS MK+816.5

### (2-d) Fmoc-D2-STag

In 1.68 L of DMF, 524 g (673 mmol) of HO-Dpm-C=NH and 206 g (3.03 mmol) of imidazole were suspended, and 0.51 L of triisopropylsilyl chloride was added dropwise thereto at 30° C. The resulting mixture was stirred for three hours, and then 5.05 L of heptane, 0.56 L of diisopropylethylamine, and 5.05 L of water were added thereto. The resulting mixture was stirred for ten minutes. Washing was performed with 0.84 L of heptane, and liquid separation and washing were performed. To the obtained upper layer, 3.37 L of 50% acetonitrile water was added, and the resulting mixture was liquid-separated and washed. To the obtained upper layer, 3.37 L of 50% acetonitrile water was added again, and the resulting mixture was liquid-separated and washed. Washing was performed with 0.84 L of heptane, and the obtained upper layer was concentrated under reduced pressure. To the obtained residue, 0.84 L of tetrahydrofuran was added, and the resulting mixture was concentrated again under reduced pressure. The obtained residue was dissolved in 0.42 L of tetrahydrofuran, 6.31 L of isopropanol was added dropwise thereto with stirring, and the resulting mixture was stirred for one hour after the dropwise addition. The process liquid in which a target product was crystallized was filtered, and the filtered product was washed with 2.0 L of isopropanol. The washed product was dried under reduced pressure at 40° C., 4.0 L of acetonitrile was added to the obtained residue, and the resulting mixture was stirred at 25° C. for one hour. The slurry was filtered. The filtered product was washed with 2.0 L of acetonitrile. Thereafter, the washed product was dried at 40° C. under reduced pressure to obtain 521 g (yield: 70.9%) of Fmoc-D2-STag. The obtained Fmoc-D2-STag was a solid.

¹H-NMR (400 MHz, CDCl3) δ 0.99-1.12 (m, 6H), 1.06 (s, 36H), 1.18-1.38 (m, 24H), 1.38-1.48 (m, 4H), 1.48-1.60 (m, 4H), 1.70-1.83 (m, 4H), 3.66 (dd, 4H), 3.93 (dd, 4H), 4.21 (br, 1H), 4.44 (d, 1H), 50.00-5.13 (br, 0.18H), 5.20-5.32 (br d, 0.82H), 5.70-5.80 (br, 0.18H), 5.82-5.92 (br d, 0.82H), 6.84 (d, 4H), 7.11 (d, 4H), 7.28-7.34 (m, 2H), 7.34-7.47 (m, 2H), 7.55-7.65 (br d, 2H), 7.70-7.85 (br d, 2H)

¹³C-NMR (100 MHz, CDCl3) δ 12.0, 18.0, 25.8, 26.0, 29.3, 29.40, 29.55 (4C), 29.62, 33.0, 63.5, 680.0, 77.2, 114.5, 119.9, 125.0, 127.0, 127.6, 128.3, 133.8, 141.3, 143 0.9, 155.5, 158.4

ESIMS MNa+1112.8

### Analysis Conditions of Fmoc-D2-STag, Dimer and Dealkylsilyl Body of TIPS2-Dpm-OH (HPLC)

Column: YMC-Triart C18

(Inner diameter: 3.0 mm, length: 100 mm, and particle diameter: 1.9 μm)

Mobile phase A: obtained by adding 0.1% v/v formic acid to a solution having a ratio of isopropanol acetonitrile:water=5:90:5

Mobile phase B: obtained by adding 0.1% v/v formic acid to isopropanol

Flow rate: 0.35 mL/min

Column temperature: 35° C.

Detection wavelength: 254 nm

Gradient conditions: 40% B (0 min)->40% B (3 min)->100% B (28 min)->100% B (40 min)->40% B (41 min)->40% B (51 min)

Analysis Conditions of HO—(CH₂)₁₁-OTIPS (HPLC)

Column: YMC-Triart C18

(Inner diameter: 3.0 mm, length: 100 mm, and particle diameter: 1.9 μm)

Mobile phase A: a solution containing 0.01M ammonium formate and having a ratio of acetonitrile:water=8:2

Mobile phase B: a solution containing 0.01M ammonium formate and having a ratio of isopropanol:water=100:1

Flow rate: 0.25 mL/min

Column temperature: 35° C.

Detection wavelength: 200 nm

Gradient conditions: 0% B (0 min)->0% B (5 min)->100% B (22 min)->100% B (27 min)->0% B (29 min)->0% B (51 min)

In Comparative Example, HO—(CH₂)₁₁-OTIPS contained as impurities in the crude purified TIPS2-Dpm-OH was carried over as it was. This component was not detected in Example. In Comparative Example, there was also an issue that TIPS2-Dpm-OH as an intermediate was decomposed by a trace amount of acid and dimerized during storage. In Example, since the production method does not pass through TIPS2-Dpm-OH, no dimer of TIPS2-Dpm-OH which is hard to separate was detected.

In the step (1-d) of Comparative Example 1, a side reaction in which the TIPS group was deprotected by an acid catalyst also occurred. In Example 1, TIPS formation was performed in the final step, and a condition of using an acid catalyst was avoided. Therefore, a dealkylsilyl body was not detected, and this issue was settled.

In Comparative Example 1, Br—(CH₂)₁₁—O-TIPS was added in an amount of 2.25 equivalents with respect to 4,4′-dihydroxybenzophenone. 0.25 equivalent of Br—(CH₂)₁₁—O-TIPS as a surplus and HO—(CH₂)₁₁—O-TIPS obtained by decomposition of the present compound were lipid-soluble, and had similar physical properties to TIPS2-Dpm-C=O and TIPS2-Dpm-OH as target products in liquid separation operation, and were difficult to separate. Both TIPS2-Dpm-C=O and TIPS2-Dpm-OH, which are intermediates with elongated side chains, were oily compounds, and therefore it was not possible to perform an operation of solidifying TIPS2-Dpm-C=O or TIPS2-Dpm-OH and washing and removing Br—(CH₂)₁₁—O-TIPS using a specific solvent. Therefore, these compounds derived from the raw materials of the side chain elongation reaction were mixed in TIPS2-Dpm-OH while maintaining a large amount of nearly 0.25 equivalent with respect to 4,4′-dihydroxybenzophenone. TIPS2-Dpm-OH contained as much as 5.1% by weight of HO—(CH₂)₁₁-OTIPS. Also when TIPS2-Dpm-OH was amidated and converted into Fmoc-D2-STag, HO—(CH₂)₁₁—O-TIPS was mixed without being removed.

In contrast, in Example 1, the intermediate HO-Dpm-C=O, in which a side chain was elongated using 11 bromo-1 undecanol, was a solid. Therefore, 11-bromo-1-undecanol was easily removed by washing HO-Dpm-C=O with an organic solvent such as methanol having high solubility of 11-bromo-1-undecanol. The content of a compound derived from 11-bromo-1-undecanol contained in Fmoc-D2-STag was 0.03% by weight. Further, unlike Comparative Example, all the intermediates were obtained as solids. Therefore, it was possible to roughly purify the intermediates by solid-liquid separation, and it was possible to improve purity of Fmoc-D2-STag as a final target product.

In Comparative Example 1, there is also an issue that TIPS2-Dpm-OH as an intermediate was decomposed by a trace amount of acid and dimerized during storage. Example 1 does not pass through TIPS2-Dpm-OH, thereby this issue was settled.

Further, in the step (1-d) of Comparative Example 1, a side reaction in which the TIPS group was deprotected by an acid catalyst also occurred. In Example 1, TIPS formation was performed in the final step, and a condition of using an acid catalyst was avoided. Therefore, this issue was settled.

As described above, the contamination amount of HO—(CH₂)₁₁-OTIPS, which is an impurity whose separation is difficult by the production method of the present invention, could be largely reduced as compared with the conventional method. In addition, by obtaining all the intermediates in the process as solids, crude purification by solid-liquid separation was made possible. In the conventional method, it is necessary to perform precise purification, for example, by column chromatography, for example, in the step of separating HO—(CH₂)₁₁-OTIPS. Therefore, in the conventional method, it is difficult to produce a large amount of alkylsilyloxy-alkyloxybenzylamine compound on an industrial scale. In contrast, in the production method of the present application, the contamination amount of HO—(CH₂)₁₁-OTIPS can be largely reduced, and an alkylsilyloxy-substituted benzylamine compound can be industrially advantageously obtained. Further, in the production method of the present application, the issue that the intermediate is decomposed by the acid catalyst, which has been an issue in the conventional method, has also been settled.

Br—(CH₂)₁₁—O-TIPS used in the conventional method is an unstable compound that is gradually decomposed at room temperature, and the method of the present invention also has an advantage of avoiding use of an unstable raw material.

1: A method for producing a ketimine compound of general formula (3): 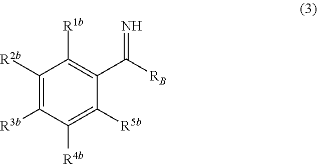
in which one to five of R^{1b} to R^{5b} each represent a hydroxyalkyloxy group having 1-16 carbon atoms, the remaining groups each represent a hydrogen atom, a halogen atom, an alkyl group having 1-4 carbon atoms, or an alkoxy group having 1-4 carbon atoms, and
R_{B} represents a hydrogen atom, a hydroxyl group, an alkoxy group having 1-6 carbon atoms, or a group of the following formula: 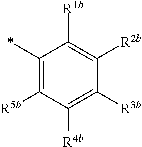
one to five of R^{1b} to R^{5b} each represent a hydroxyalkyloxy group having 1-16 carbon atoms, and the remaining groups each represent a hydrogen atom, a halogen atom, an alkyl group having 1-4 carbon atoms, or an alkoxy group having 1-4 carbon atoms, or when R_{B} represents a structure of this formula, R^{5b} may form an ether bond (—O—) together with R^{5b} in formula (3). *. represents a bonding site to R_{B}.)),
wherein a silazane compound is subjected to a reaction with a benzoyl compound of general formula (2): 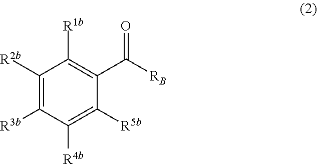
in which one to five of R^{1b} to R^{5b} each represent a hydroxyalkyloxy group having 1-16 carbon atoms, the remaining groups each represent a hydrogen atom, a halogen atom, an alkyl group having 1-4 carbon atoms, or an alkoxy group having 1-4 carbon atoms, and
R_{B} represents a hydrogen atom, a hydroxy group, an alkoxy group having 1-6 carbon atoms, or a group of 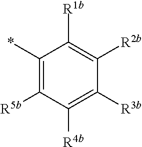
one to five of R^{1b} to R^{5b} each represent a hydroxyalkyloxy group having 1-16 carbon atoms, and the remaining groups each represent a hydrogen atom, a halogen atom, an alkyl group having 1-4 carbon atoms, or an alkoxy group having 1-4 carbon atoms, or when R_{B} represents a structure of this formula, R^{5b} may form an ether bond (—O—) together with R^{5b} in formula (2) * represents a bonding site to R_{B}.,
and then the resulting product is subjected to a reaction with an acid or a base.

2: A method for producing a benzylamine compound of general formula (4): 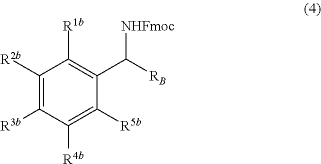
in which one to five of R^{1b} to R^{5b} each represent a hydroxyalkyloxy group having 1-16 carbon atoms, the remaining groups each represent a hydrogen atom, a halogen atom, an alkyl group having 1-4 carbon atoms, or an alkoxy group having 1-4 carbon atoms, and
R_{B} represents a hydrogen atom, a hydroxy group, an alkoxy group having 1-6 carbon atoms, or a group of the following formula: 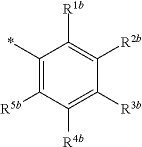
one to five of R^{1b} to R^{5b} each represent a hydroxyalkyloxy group having 1-16 carbon atoms, and the remaining groups each represent a hydrogen atom, a halogen atom, an alkyl group having 1-4 carbon atoms, or an alkoxy group having 1-4 carbon atoms, or when R_{B} represents a structure of this formula, R^{5b} may form an ether bond (—O—) together with R^{5b} in formula (4). * . represents a bonding site to R_{B}.,
wherein a silazane compound is subjected to a reaction with a benzoyl compound of general formula (2): 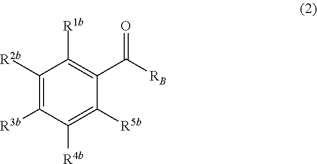
in which one to five of R^{1b} to R^{5b} each represent a hydroxyalkyloxy group having 1-16 carbon atoms, the remaining groups each represent a hydrogen atom, a halogen atom, an alkyl group having 1-4 carbon atoms, or an alkoxy group having 1-4 carbon atoms, and
R_{B} represents a hydrogen atom, a hydroxy group, an alkoxy group having 1-6 carbon atoms, or a group of 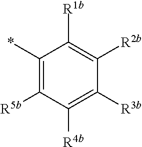
one to five of R^{1b} to R^{5b} each represent a hydroxyalkyloxy group having 1-16 carbon atoms, and the remaining groups each represent a hydrogen atom, a halogen atom, an alkyl group having 1-4 carbon atoms, or an alkoxy group having 1-4 carbon atoms, or when R_{B} represents a structure of this formula, R^{5b} may form an ether bond (—O—) together with R^{5b} in formula (2). * represents a bonding site to R_{B}.,
then causing the resulting product to react with an acid or a base to obtain a ketimine compound of general formula (3): 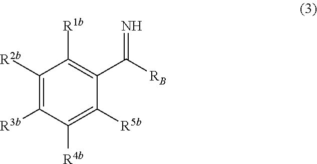
in which one to five of R^{1b} to R^{5b} each represent a hydroxyalkyloxy group having 1-16 carbon atoms, the remaining groups each represent a hydrogen atom, a halogen atom, an alkyl group having 1-4 carbon atoms, or an alkoxy group having 1-4 carbon atoms, and
R_{B} represents a hydrogen atom, a hydroxy group, an alkoxy group having 1-6 carbon atoms, or a group of the following formula: 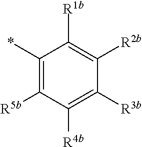
one to five of R^{1b} to R^{5b} each represent a hydroxyalkyloxy group having 1-16 carbon atoms, and the remaining groups each represent a hydrogen atom, a halogen atom, an alkyl group having 1-4 carbon atoms, or an alkoxy group having 1-4 carbon atoms, or when R_{B} represents a structure of this formula, R^{5b} may form an ether bond (—O—) together with R^{5b} in formula (3). *. represents a bonding site to R_{B}., and
causing the obtained ketimine compound to react with a reducing agent and an Fmoc-forming agent.

3: A method for producing an alkylsilyloxy-substituted benzylamine compound of general formula (5): 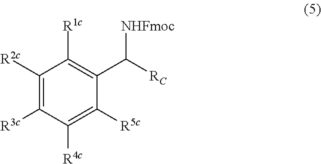
in which one to five of R^{1c} to R^{5c} each represent an alkyloxy group having 1-29 carbon atoms and having 1-3 alkylsilyloxy groups as substituents, the remaining groups each represent a hydrogen atom, a halogen atom, an alkyloxy group having 1-4 carbon atoms, or an alkoxy group having 1-4 carbon atoms, and
R_{C} represents a hydrogen atom, a hydroxy group, an alkoxy group having 1-6 carbon atoms, or a group of the following formula: 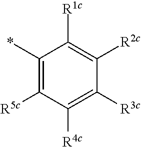
one to five of R^{1c} to R^{5c} each represent an alkyloxy group having 1-29 carbon atoms and having 1-3 alkylsilyloxy groups as substituents, and the remaining groups each represent a hydrogen atom, a halogen atom, an alkyl group having 1-4 carbon atoms, or an alkoxy group having 1-4 carbon atoms, or when R_{C} represents a structure of this formula, R^{5c} may form an ether bond (—O—) together with R^{5c} in formula (5). * represents a bonding site to R_{C}.)),
wherein a silazane compound is subjected to a reaction with a benzoyl compound of general formula (2): 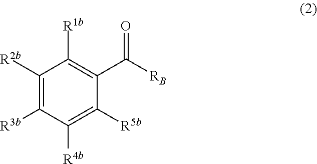
in which one to five of R^{1b} to R^{5b} each represent a hydroxyalkyloxy group having 1-16 carbon atoms, the remaining groups each represent a hydrogen atom, a halogen atom, an alkyl group having 1-4 carbon atoms, or an alkoxy group having 1-4 carbon atoms, and
R_{B} represents a hydrogen atom, a hydroxy group, an alkoxy group having 1-6 carbon atoms, or a group of 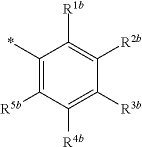
one to five of R^{1b} to R^{5b} each represent a hydroxyalkyloxy group having 1-16 carbon atoms, and the remaining groups each represent a hydrogen atom, a halogen atom, an alkyl group having 1-4 carbon atoms, or an alkoxy group having 1-4 carbon atoms, or when R_{B} represents a structure of this formula, R^{5b} may form an ether bond (—O—) together with R^{5b} in formula (2). *. represents a bonding site to R_{B}.,
then causing the resulting product to react with an acid or a base to obtain a ketimine compound of general formula (3): 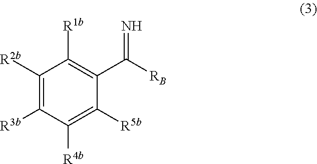
in which one to five of R^{1b} to R^{5b} each represent a hydroxyalkyloxy group having 1-16 carbon atoms, the remaining groups each represent a hydrogen atom, a halogen atom, an alkyl group having 1-4 carbon atoms, or an alkoxy group having 1-4 carbon atoms, and
R_{B} represents a hydrogen atom, a hydroxy group, an alkoxy group having 1-6 carbon atoms, or a group of the following formula: 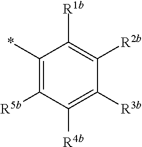
one to five of R^{1b} to R^{5b} each represent a hydroxyalkyloxy group having 1-16 carbon atoms, and the remaining groups each represent a hydrogen atom, a halogen atom, an alkyl group having 1-4 carbon atoms, or an alkoxy group having 1-4 carbon atoms, or when R_{B} represents a structure of this formula, R^{5b} may form an ether bond (—O—) together with R^{5b} in formula (3). * . represents a bonding site to R_{B}.,
causing the obtained ketimine compound to react with a reducing agent and an Fmoc-forming agent to obtain a benzylamine compound of general formula (4): 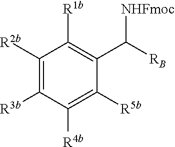
in which one to five of R^{1b} to R^{5b} each represent a hydroxyalkyloxy group having 1-16 carbon atoms, the remaining groups each represent a hydrogen atom, a halogen atom, an alkyl group having 1-4 carbon atoms, or an alkoxy group having 1-4 carbon atoms, and
R_{B} represents a hydrogen atom, a hydroxy group, an alkoxy group having 1-6 carbon atoms, or a group of the following formula: 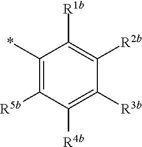
one to five of R^{1b} to R^{5b} each represent a hydroxyalkyloxy group having 1-16 carbon atoms, and the remaining groups each represent a hydrogen atom, a halogen atom, an alkyl group having 1-4 carbon atoms, or an alkoxy group having 1-4 carbon atoms, or when R_{B} represents a structure of this formula, R^{5b} may form an ether bond (—O—) together with R^{5b} in formula (4). * . represents a bonding site to R_{B}.)),
and then causing the benzylamine compound to react with an alkylsilylating agent.

4: The production method according to claim 1, wherein the benzoyl compound of general formula (2) is obtained by causing a halogenated alcohol to react with a compound of general formula (1): 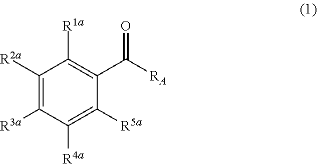
in which one to five of R^{1a} to R^{5a} each represent a hydroxy group, the remaining groups each represent a hydrogen atom, a halogen atom, an alkyl group having 1-4 carbon atoms, or an alkoxy group having 1-4 carbon atoms, and
R_{A} represents a hydrogen atom, a hydroxy group, an alkoxy group having 1-6 carbon atoms, or a group of the following formula: 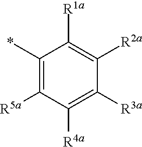
(one to five of R^{1a} to R^{5a} each represent a hydroxy group, and the remaining groups each represent a hydrogen atom, a halogen atom, an alkyl group having 1-4 carbon atoms, or an alkoxy group having 1-4 carbon atoms, or when R_{A} represents a structure of this formula, R^{5a} may form an ether bond (—O—) together with R^{5a} in formula (1). * represents a bonding site to R_{A}.

5: A compound of general formula (6): 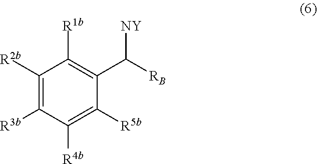
in which one to five of R^{1b} to R^{5b} each represent a hydroxyalkyloxy group having 1-16 carbon atoms, the remaining groups each represent a hydrogen atom, a halogen atom, an alkyl group having 1-4 carbon atoms, or an alkoxy group having 1-4 carbon atoms, and
R_{B} represents a hydrogen atom, a hydroxy group, an alkoxy group having 1-6 carbon atoms, or a group of the following formula: 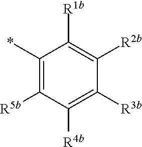
one to five of R^{1b} to R^{5b} each represent a hydroxyalkyloxy group having 1-16 carbon atoms, and the remaining groups each represent a hydrogen atom, a halogen atom, an alkyl group having 1-4 carbon atoms, or an alkoxy group having 1-4 carbon atoms, or when R_{B} represents a structure of this formula, R^{5b} may form an ether bond (—O—) together with R^{5b} in formula (6). *. represents a bonding site to R_{B}.), and
NY represents an imino group (═NH) or NHFmoc.

6: A method for producing an alkylsilyloxy-substituted benzylamine compound of general formula (5): 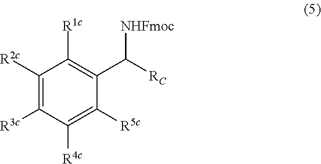
in which one to five of R^{1c} to R^{5c} each represent an alkyloxy group having 1-29 carbon atoms and having 1-3 alkylsilyloxy groups as substituents, the remaining groups each represent a hydrogen atom, a halogen atom, an alkyloxy group having 1-4 carbon atoms, or an alkoxy group having 1-4 carbon atoms, and
R_{C} represents a hydrogen atom, a hydroxy group, an alkoxy group having 1-6 carbon atoms, or a group of the following formula: 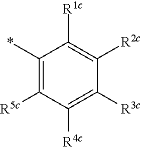
one to five of R^{1c} to R^{5c} each represent an alkyloxy group having 1-29 carbon atoms and having 1-3 alkylsilyloxy groups as substituents, and the remaining groups each represent a hydrogen atom, a halogen atom, an alkyl group having 1-4 carbon atoms, or an alkoxy group having 1-4 carbon atoms, or when R_{c} represents a structure of this formula, R^{5c} may form an ether bond (—O—) together with R^{5c} in formula (5). * represents a bonding site to R_{C}.,
wherein an alkylsilylating agent is subjected to a reaction with a benzylamine compound of general formula (4): 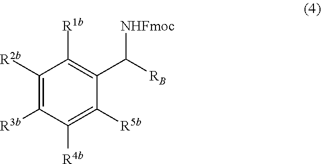
in which one to five of R^{1b} to R^{5b} each represent a hydroxyalkyloxy group having 1-16 carbon atoms, the remaining groups each represent a hydrogen atom, a halogen atom, an alkyl group having 1-4 carbon atoms, or an alkoxy group having 1-4 carbon atoms, and
R_{B} represents a hydrogen atom, a hydroxy group, an alkoxy group having 1-6 carbon atoms, or a group of the following formula: 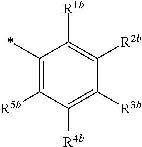
one to five of R^{1b} to R^{5b} each represent a hydroxyalkyloxy group having 1-16 carbon atoms, and the remaining groups each represent a hydrogen atom, a halogen atom, an alkyl group having 1-4 carbon atoms, or an alkoxy group having 1-4 carbon atoms, or when R_{B} represents a structure of this formula, R^{5b} may form an ether bond (—O—) together with R^{5b} in formula (4). * . represents a bonding site to R_{B}.

7: A method for producing an alkylsilyloxy-substituted benzylamine compound of general formula (5): 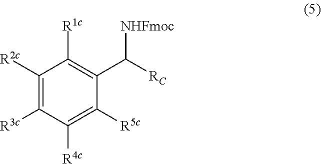
in which one to five of R^{1c} to R^{5c} each represent an alkyloxy group having 1-29 carbon atoms and having 1-3 alkylsilyloxy groups as substituents, the remaining groups each represent a hydrogen atom, a halogen atom, an alkyloxy group having 1-4 carbon atoms, or an alkoxy group having 1-4 carbon atoms, and
R_{C} represents a hydrogen atom, a hydroxy group, an alkoxy group having 1-6 carbon atoms, or a group of the following formula: 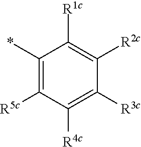
one to five of R^{1c} to R^{5c} each represent an alkyloxy group having 1-29 carbon atoms and having 1-3 alkylsilyloxy groups as substituents, and the remaining groups each represent a hydrogen atom, a halogen atom, an alkyl group having 1-4 carbon atoms, or an alkoxy group having 1-4 carbon atoms, or when R_{c} represents a structure of this formula, R^{5c} may form an ether bond (—O—) together with R^{5c} in formula (5). * represents a bonding site to R_{C}.,
wherein a reducing agent and an Fmoc-forming agent are subjected to a reaction with a ketimine compound of general formula (3): 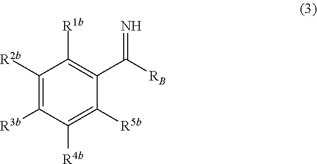
in which one to five of R^{1b} to R^{5b} each represent a hydroxyalkyloxy group having 1-16 carbon atoms, the remaining groups each represent a hydrogen atom, a halogen atom, an alkyl group having 1-4 carbon atoms, or an alkoxy group having 1-4 carbon atoms, and
R_{B} represents a hydrogen atom, a hydroxy group, an alkoxy group having 1-6 carbon atoms, or a group of the following formula: 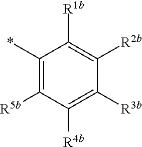
one to five of R^{1b} to R^{5b} each represent a hydroxyalkyloxy group having 1-16 carbon atoms, and the remaining groups each represent a hydrogen atom, a halogen atom, an alkyl group having 1-4 carbon atoms, or an alkoxy group having 1-4 carbon atoms, or when R_{B} represents a structure of this formula, R^{5b} may form an ether bond (—O—) together with R^{5b} in formula (3). * . represents a bonding site to R_{B}.)) to obtain a benzylamine compound of general formula (4): 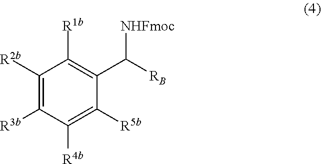
in which one to five of R^{1b} to R^{5b} each represent a hydroxyalkyloxy group having 1-16 carbon atoms, the remaining groups each represent a hydrogen atom, a halogen atom, an alkyl group having 1-4 carbon atoms, or an alkoxy group having 1-4 carbon atoms, and
R_{B} represents a hydrogen atom, a hydroxy group, an alkoxy group having 1-6 carbon atoms, or a group of the following formula: 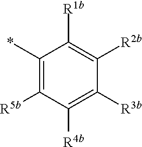
one to five of R^{1b} to R^{5b} each represent a hydroxyalkyloxy group having 1-16 carbon atoms, and the remaining groups each represent a hydrogen atom, a halogen atom, an alkyl group having 1-4 carbon atoms, or an alkoxy group having 1-4 carbon atoms, or when R_{B} represents a structure of this formula, R^{5b} may form an ether bond (—O—) together with R^{5b} in formula (4). *. represents a bonding site to R_{B}.,
and then causing the benzylamine compound to react with an alkylsilylating agent.
